(19) United States
(12) Patent Application Publication
(10) Pub. No.: US 2020/0040093 A1
(43) Pub. Date: Feb. 6, 2020

(54) COMPOSITIONS AND METHODS FOR NON-MYELOABLATIVE CONDITIONING

(71) Applicants: President and Fellows of Harvard College, Cambridge, MA (US); The General Hospital Corporation, Boston, MA (US)

(72) Inventors: David T. Scadden, Weston, MA (US); Rahul Palchaudhuri, Cambridge, MA (US)

(21) Appl. No.: 16/341,437

(22) PCT Filed: Oct. 13, 2017
(86) PCT No.: PCT/US2017/056675

§ 371 Date: Apr. 11, 2019

Related U.S. Application Data
(60) Provisional application No. 62/407,946, filed on Oct. 13, 2016.

(51) Int. Cl. C07K 16/28; A61K 47/68

(52) U.S. Cl. CPC C07K 16/2896; C07K 16/2803; C07K 16/2854; C07K 16/2881; A61K 47/6819; C07K 2317/31; A61K 47/6829; C07K 2317/77; C07K 2317/24; C07K 2317/622; A61K 47/6831

(57) ABSTRACT

Disclosed herein are non-myeloablative antibody-toxin conjugates and compositions that target cell surface markers and related methods of their use to effectively conditioning a subject's tissues (e.g., bone marrow tissue) prior to engraftment or transplant. The compositions and methods disclosed herein may be used to condition a subject's tissues in advance of, for example, hematopoietic stem cell transplant and advantageously such compositions and methods do not cause the toxicities that are commonly associated with traditional conditioning methods.

### RELATED APPLICATION

This application claims the benefit of U.S. Provisional Application No. 62/407,946, filed on Oct. 13, 2016. The entire teachings of the above application are incorporated herein by reference.

### GOVERNMENT FUNDING

This invention was made with government support under HL097794, awarded by the National Institutes of Health. The government has certain rights in the invention.

### BACKGROUND OF THE INVENTION

Hematopoietic stem cell transplant (HSCT) is primarily indicated to treat malignancies and requires a conditioning of the subject's tissues (e.g., bone marrow tissue) prior to engraftment. HSCT indications and hemoglobinopathies include, for example, sickle cell anemia, beta thalassemias, Fanconi anemia, Wiskott-Aldrich syndrome, adenosine deaminase SCID (ADA SCID), metachromatic leukodystrophy and HIV/AIDS; the list of indications will continue to expand with improvement in gene editing technologies. In certain instances, 20% engraftment of transplanted cells may alleviate or cure the disease.

Current non-targeted conditioning methods, which include, for example, irradiation (e.g., total body irradiation or TBI) and DNA alkylating/modifying agents, are highly toxic to multiple organ systems, hematopoietic and non-hematopoietic cells and the hematopoietic microenvironment. These harsh conditioning regimens effectively kill the host subject's immune and niche cells and adversely affect multiple organ systems, frequently leading to life-threatening complications.

To fully realize the curative potential of HSCT, the development of mild-conditioning regimens that avoid undesirable toxicity is essential. Needed are novel, preferably non-myeloablative, compositions and methods that may be used to condition a subject's tissues (e.g., bone marrow tissues), while lessening undesirable toxicity and minimizing the incidence of serious adverse reactions. Also needed are novel therapies that can selectively ablate an endogenous hematopoietic stem cell population in a target tissue, while minimizing or eliminating the effects of such therapies on non-targeted cells and tissues, such as platelets, white blood cells and red blood cells. Also needed are assays and methods for identifying agents that can selectively deplete or ablate an endogenous hematopoietic stem cell population.

### SUMMARY OF THE INVENTION

Disclosed herein are methods and compositions that are useful for ablating selected cell populations and conditioning a subject's tissues for engraftment or transplant, as well as assays and methods of identifying candidate agents that are useful for conditioning a subject's tissues for engraftment or transplant. In certain embodiments, the methods and compositions disclosed herein are non-myeloablative. Also disclosed are methods of delivering a toxin to a cell, e.g., by targeting one or more markers (e.g., the cell surface CD45 marker), such that the toxin is internalized; such methods are useful for effectively conditioning a subject for engraftment or transplant (e.g., conditioning a human subject for hematopoietic stem cell transplant).

Advantageously, the methods, assays and compositions disclosed herein do not cause the toxicities that have generally been associated with traditional conditioning methods, such as irradiation. For example, relative to traditional conditioning regimens, in certain embodiments the compositions and methods disclosed herein do not induce neutropenia, thrombocytopenia and/or anemia, yet result in a stable, mixed chimerism that is of therapeutic relevance. Such compositions and methods may be used, for example, to correct, cure or otherwise ameliorate one or more diseases in an affected subject (e.g., the methods and compositions disclosed herein may be used to correct or cure HIV, AIDS, or hemoglobinopathies, such as sickle cell anemia and Fanconi anemia).

In certain embodiments, disclosed herein are methods of conditioning a subject or a subject's target tissues for engraftment, such methods comprising a selective depletion or ablation of an endogenous stem cell (e.g., hematopoietic stem cell) or progenitor cell population in a target tissue of the subject by administering to the subject an effective amount of an agent coupled (e.g., functionally coupled) to a toxin; wherein the toxin is internalized by the endogenous stem cell population, thereby depleting or ablating the endogenous stem cell population in the target tissue and conditioning the subject for engraftment of a transplanted cell or cell population. In certain embodiments the agent is selected from the group consisting of an antibody and a ligand.

Also disclosed herein are methods of engrafting stem cells in a subject, such methods comprising: (a) administering to the subject an effective amount of an agent coupled to a toxin, wherein the toxin is internalized by an endogenous stem cell (e.g., hematopoietic stem cell) or progenitor cell population, thereby selectively depleting or ablating the endogenous stem cell population in a target tissue of the subject; and (b) administering a stem cell population to the target tissue of the subject, wherein the administered stem cell population engrafts in the target tissue of the subject.

In certain aspects, also disclosed herein are methods of treating a stem cell disorder in a subject, such methods comprising: (a) administering to the subject an effective amount of an agent coupled (e.g., functionally coupled) to a toxin, wherein the toxin is internalized by an endogenous stem cell (e.g., hematopoietic stem cell) or progenitor cell population in a target tissue of the subject, thereby depleting or ablating the endogenous stem cell or progenitor cell population in the target tissue of the subject; and (b) administering a stem cell population to the target tissue of the subject, wherein the administered stem cell population engrafts in the target tissue of the subject. In some embodiments, the stem cell population is administered to the target tissues of the subject after the immunotoxin has cleared or dissipated from the subject's target tissues.

In certain embodiments, the inventions disclosed herein are directed to methods of selectively depleting or ablating an endogenous hematopoietic stem cell (HSC) or progenitor cell population in a target tissue of a subject, the methods comprising administering to the subject an effective amount (e.g., about 1.5-3.0 mg/kg) of an agent coupled to a toxin; wherein the agent selectively binds to CD45 and the toxin is internalized by the endogenous HSC or progenitor cell population, thereby depleting or ablating the endogenous HSC or progenitor cell population in the target tissue.

In some embodiments, the inventions disclosed herein are directed to methods of selectively depleting or ablating an endogenous hematopoietic stem cell or progenitor cell population in a target tissue of a subject, the methods comprising administering to the subject an effective amount of an agent coupled (e.g., functionally coupled) to a toxin; wherein the agent selectively binds to CD45 and the toxin is internalized by the endogenous HSC or progenitor cell population, thereby depleting or ablating the endogenous HSC or progenitor cell population in the target tissue.

Also disclosed herein are methods of selectively ablating an endogenous stem cell (e.g., hematopoietic stem cells) or progenitor cell population in a target tissue of a subject, the methods comprising: administering to the subject an effective amount of an internalizing antibody which specifically or selectively binds to CD45 and is coupled to a toxin and thereby ablating the endogenous stem cell population in the target tissue.

In certain embodiments, disclosed herein are methods of stem cell transplant (e.g., hematopoietic stem cell transplant), such methods comprising: administering to a subject an effective amount of an internalizing antibody which specifically or selectively binds to CD45 and is coupled to a toxin and thereby ablating an endogenous stem cell population in a target tissue; and administering an exogenous stem cell population in the target tissue of the subject.

In certain aspects, also disclosed are methods of treating or curing a hemoglobinopathy (e.g., sickle cell anemia) in a subject, the methods comprising: administering to the subject an effective amount of an internalizing antibody that specifically or selectively binds to CD45 and is coupled to a toxin and thereby ablating an endogenous stem cell (e.g., hematopoietic stem cell) or progenitor cell population in a target tissue of the subject; followed by a step of administering an exogenous stem cell population to the target tissue of the subject. In some embodiments, the exogenous stem cell population is administered to the target tissues of the subject after the immunotoxin (e.g., an anti-CD45-SAP immunotoxin) has cleared or dissipated from the subject's target tissues.

In certain aspects, the agents disclosed herein selectively target a population of cells of the target tissues. For example, in certain embodiments, such an agent (e.g., an antibody or ligand) may be internalized by a targeted hematopoietic stem cell upon binding of such agent to a cell surface protein expressed by the hematopoietic stem cell. Cell surface proteins expressed by the cells of the target tissue (e.g., hematopoietic stem cells residing in the bone marrow stem cell niche) thus provide a means of targeting, in some instances discriminately, the immunotoxins disclosed herein to a population of cells expressing that protein. In some instances, the expression of the protein is restricted to a specific cell population, and the protein can be used as a target to deliver the immunotoxin selectively to that cell population while not affecting or minimally affecting the cell populations which don't express the protein (e.g., non-target tissues or off-target tissues of the subject). Alternatively, the expression of the cell surface protein to be targeted by the immunotoxin is not restricted to a specific cell population; in these instances it is possible to use a different moiety to restrict delivery of the immunotoxin to only a subset of the cell population expressing the cell surface protein target. For example, in the context of a bispecific antibody, one specificity can be for the target cell surface protein and the other specificity can be for a marker having expression restricted to the cell population of choice.

In certain embodiments, the cells of a subject's target tissues comprise an endogenous stem cell population, such as for example, endogenous hematopoietic stem cells and/or progenitor cells residing in the target tissue. In certain aspects, the hematopoietic stem cells or progenitor cells express one or more markers that may be used to selectively target the agents comprising the immunotoxin compositions disclosed herein to the cells of the subject's target tissues.

Any markers that are capable of being used to discriminate the target cell population from the population of non-targeted cells, including any of the markers described herein, can be targeted by the agents that comprise the immunotoxins described herein for delivery of toxin to the cell population. For example, in certain aspects of the present inventions, an agent that comprises the immunotoxin composition may selectively bind to one or more cell surface markers expressed by the cells of the target tissues (e.g., a CD45-SAP immunotoxin may selectively bind to hematopoietic stem cells having cell surface expression of the CD45 marker). In certain embodiments, the targeted hematopoietic stem cells or progenitor cells express one or more markers that may be targeted and to which the immunotoxin selectively or preferentially binds, such markers selected from the group of markers consisting of HLA-DR, CD11a, CD18, CD34, CD41/61, CD43, CD45, CD49d (VLA-4), CD49f (VLA-6), CD51, CD58, CD71, CD84, CD97, CD134, CD162, CD166, CD184 (CXCR4), CD205 and CD361. In certain embodiments, the targeted cells (e.g., the hematopoietic stem cells or progenitor cells) in the target tissue express one or more markers that may be targeted and to which the immunotoxin selectively or preferentially binds, such markers selected from the group of markers consisting of: CD13, CD33, CD34, CD44, CD45, CD49d: VLA-4, CD49f: VLA-6, CD59, CD84, CD93, CD105: Endoglin, CD123: IL-3R, CD126: IL-6R, CD135: Flt3 receptor, CD166: ALCAM, CD184: CXCR4, Prominin 2, Erythropoietin R, CD244, Tie1, Tie2, G-CSFR or CSF3R, IL-1R, gp130, Leukemia inhibitory factor Receptor, oncostatin M receptor, Embigin and IL-18R.

In certain embodiments, the targeted cells (e.g., the hematopoietic stem cells or progenitor cells) in the target tissue express one or more markers that may be targeted and to which the immunotoxin selectively or preferentially binds, such markers selected from the group of markers consisting of: HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L. In certain embodiments, the targeted cells (e.g., the hematopoietic stem cells or progenitor cells) in the target tissue express one or more markers that may be targeted and to which the immunotoxin selectively or preferentially binds, such markers selected from the group of markers consisting of: CD51/61, CD72, CD45RA, CD107a, CD45RB, CD7, CD13, CD132 and CD321.

In still other embodiments, the targeted cells (e.g., hematopoietic stem cells or progenitor cells) in the target tissue express one or more markers that may be targeted and to which the agents that comprise the immunotoxin selectively bind, such markers as CD45. For example, in some embodiments, the hematopoietic stem cells or progenitor cells express CD45. Similarly, in some embodiments, the hematopoietic stem cells or progenitor cells express CD34.

In certain embodiments, the marker is selected from the group consisting of HLA-DR, CD11a, CD18, CD34, CD41/61, CD43, CD45, CD47, CD58, CD71, CD84, CD97, CD162, CD166, CD205 and CD361. In certain embodiments, the targeted cells comprise human hematopoietic stem cells expressing one or more markers that may be targeted and to which the agents that comprise the immunotoxin bind, such markers selected from the group consisting of CD7, CDw12, CD13, CD15, CD19, CD21, CD22, CD29, CD30, CD33, CD34, CD36, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD99, CD104, CD105, CD109, CD111, CD112, CD114, CD115, CD123, CD124, CD126, CD127, CD130, CD131, CD135, CD138, CD151, CD157, CD162, CD164, CD168, CD172a, CD173, CD174, CD175, CD175s, CD176, CD183, CD191, CD200, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349 and CD350.

In certain embodiments, the targeted cells comprise human hematopoietic stem cells expressing one or more markers that may be targeted and to which the agents that comprise the immunotoxin bind, such markers selected from the group consisting of CD11a, CD18, CD37, CD47, CD52, CD58, CD62L, CD69, CD74, CD97, CD103, CD132, CD156a, CD179a, CD179b, CD184, CD232, CD244, CD252, CD302, CD305, CD317 and CD361.

In certain aspects, the targeted cells comprise human hematopoietic stem cells or progenitor cells expressing one or more markers selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L.

In certain aspects, the targeted cells comprise human hematopoietic stem cells or progenitor cells expressing one or more markers selected from the group consisting of CD51/61, CD72, CD45RA, CD107a, CD45RB, CD7, CD13, CD132 and CD321.

In certain embodiments, the endogenous cells (e.g., HSCs or progenitor cells) express one or more markers, and the administered agent (e.g., an antibody-toxin conjugate) selectively binds to the one or more markers or a fragment or epitope thereof. In certain aspects the methods disclosed herein specifically or discriminatorily target or are directed towards the subject's target tissues, while not affecting or minimally affecting the non-target tissues or off-target tissues (e.g., the thymus) of the subject. In certain embodiments, the methods and compositions disclosed herein do not deplete or ablate endogenous neutrophils or myeloid cells. In certain embodiments, the methods and compositions disclosed herein cause an increase in mature endogenous neutrophils. In certain aspects, the methods and compositions disclosed herein do not deplete or ablate endogenous platelets. In still other embodiments, the methods and compositions disclosed herein do not induce anemia in the subject.

In certain embodiments, the markers are internalizing. For example, upon binding of the agent to an internalizing marker (e.g., a cell surface receptor), the composition is internalized by the cell expressing such marker.

In some embodiments, the marker is not internalizing. For example, in such embodiments, a first marker may be used as a means of discriminately targeting a cell population, while a second marker may be targeted to effectuate the internalization of the immunotoxin composition intracellularly.

The immunotoxin compositions disclosed herein comprise an agent to facilitate the selective delivery of such compositions to a population of cells in the target tissues (e.g., hematopoietic stem cells of the bone marrow stem cell niche). In some embodiments, the agents disclosed herein comprise an antibody (e.g., a monoclonal antibody). In some embodiments the antibody is a blocking antibody or an antagonist antibody. In some embodiments the antibody is not a blocking antibody or an antagonist antibody. In certain embodiments, the agents disclosed comprise a ligand. In certain aspects, the agent selectively binds to CD45. In certain aspects, the agent is a CD45 antagonist. Alternatively, in certain embodiments the agent is not a CD45 antagonist. In some embodiments, the toxin is internalized by a cell expressing CD45 following binding of the agent to an epitope of the CD45 cell surface marker.

In certain aspects, the agent is antibody clone 104. In certain embodiments, the agent is antibody clone 30F11. In certain aspects, the agent is antibody clone 3C11. In certain embodiments, the agent is antibody clone MEM-28. In certain embodiments, the agent is antibody clone HI30. In certain embodiments, the agent is antibody clone 581. In certain embodiments, the agent is antibody clone 4H11. In certain aspects, the agent is an antibody selected from the group consisting of clone L243, clone TS2/4, clone TS1/18, clone 581, clone 4H11, clone A2A9/6, clone CD43-10G7, clone BHPT-1, clone orb12060, clone 2D1, clone CC2C6, clone TS2/9, clone CY1G4, clone OKT9, clone CD84.1.21, clone VIM3b, clone A3C6E2, clone EMK08, clone TMP4, clone KPL-1, clone 3a6, clone HD83 and clone MEM-216.

In certain embodiments, the agent comprises an antibody selected from the group consisting of clone 23C6, clone J4-117, clone HI100, clone H4A3, clone MT4, clone M-T701, clone WM15, clone TUGh4 and clone M.AB.F11. In certain aspects, the agent comprises an antibody selected from the group consisting of clone TU39, clone TU99, clone N6B6, clone TU41, clone UM7F8, clone H5C6, clone G44-26, clone G46-2.6, clone HECA-452, clone CBR-1C2/2.1, clone 1C3, clone EBA-1, clone HIM6, clone p282 (H19), clone AK-4, clone CSLEX1, clone G28-8, clone 11G7, clone VC5, clone 28D4, clone 3A6, clone 2D7/CCR5, clone SN2, clone TU169, clone WM59, clone GHI/75, clone 9F5, clone HIP2, clone FN50, clone KPL-1, clone 1G10, clone M-A712, clone B6H12, clone VIM3b, clone MG38, clone G46-6 (L243), clone 581, clone 9F10, clone 12G5, clone 2G7, clone TU145, clone G43-25B and clone Dreg 56.

In certain embodiments, the agent is an antibody comprising a complementarity determining region that is the same as the complementarity determining region for one or more antibodies selected from the group consisting of L243, clone TS2/4, clone TS1/18, clone 581, clone 4H11, clone A2A9/6, clone CD43-10G7, clone BHPT-1, clone orb12060, clone 2D1, clone CC2C6, clone TS2/9, clone CY1G4, clone OKT9, clone CD84.1.21, clone VIM3b, clone A3C6E2, clone EMK08, clone TMP4, clone KPL-1, clone 3a6, clone HD83 and clone MEM-216. In certain embodiments, the agent is an antibody that binds to the same epitope as one or more antibodies selected from the group consisting of L243, clone TS2/4, clone TS1/18, clone 581, clone 4H11, clone A2A9/6, clone CD43-10G7, clone BHPT-1, clone orb12060, clone 2D1, clone CC2C6, clone TS2/9, clone CY1G4, clone OKT9, clone CD84.1.21, clone VIM3b, clone A3C6E2, clone EMK08, clone TMP4, clone KPL-1, clone 3a6, clone HD83 and clone MEM-216. In certain aspects, the agent comprises an antibody that selectively recognizes and/or binds to the CD34 marker (e.g., clone 581 or clone 4H11). In certain aspects, the agent comprises an antibody that selectively recognizes and/or binds to the CD45 marker (e.g., clone MEM-28 or clone HI30). In some embodiments, agent comprises an antibody, and wherein the antibody comprises a complementarity determining region that is the same as the complementarity determining region for one or more antibodies selected from the group consisting of clone 23C6, clone J4-117, clone HI100, clone H4A3, clone MT4, clone M-T701, clone WM15, clone TUGh4 and clone M.AB.F11. In certain aspects of the present inventions, the agent comprises an antibody, and wherein the antibody comprises a complementarity determining region that is the same as the complementarity determining region for one or more antibodies selected from the group consisting of clone TU39, clone TU99, clone N6B6, clone TU41, clone UM7F8, clone H5C6, clone G44-26, clone G46-2.6, clone HECA-452, clone CBR-1C2/2.1, clone 1C3, clone EBA-1, clone HIM6, clone p282 (H19), clone AK-4, clone CSLEX1, clone G28-8, clone 11G7, clone VC5, clone 28D4, clone 3A6, clone 2D7/CCR5, clone SN2, clone TU169, clone WM59, clone GHI/75, clone 9F5, clone HIP2, clone FN50, clone KPL-1, clone 1G10, clone M-A712, clone B6H12, clone VIM3b, clone MG38, clone G46-6 (L243), clone 581, clone 9F10, clone 12G5, clone 2G7, clone TU145, clone G43-25B and clone Dreg 56.

In certain aspects of any of the embodiments set forth herein, the agent is or comprises a humanized antibody.

In certain embodiments, the agent is a ligand. For example, in certain embodiments the ligand may be selected from the group of ligands consisting of CXCL12: Stromal derived factor 1 (SDF1), Angiopoietin 1 to 4 (Ang1, Ang2, Ang3, Ang4), TPO (thrombopoietin), Erythropoietin, FLT3L, VLA4, VLA6, IL-1, IL-3, IL-6, IL-18, G-CSF, Oncostatin M and LIF.

In certain embodiments, the agent is coupled to a toxin (e.g., saporin). In certain aspects, the agents (e.g., antibodies) disclosed herein are characterized as being internalizing. In certain aspects, such agents are internalized by a cell expressing a marker or moiety (e.g., a cell surface marker or antigen) to which the agent binds (including, but not limited to, CD45) following binding of such agent (e.g., antibody or ligand).

In some embodiments, the toxin is internalized by receptor-mediated internalization. In certain aspects, the toxins disclosed herein are internalized by the endogenous stem cell population at a rate of at least about 10% (e.g., over about 24 hours). In certain aspects, the toxins disclosed herein are internalized by the endogenous stem cell population at a rate of at least about 50% (e.g., over about 24 hours). In yet other embodiments, the toxins disclosed herein are internalized by the endogenous stem cell population at a rate of at least about 90% (e.g., over about 24 hours).

The methods disclosed herein may be practiced using any suitable toxin. In certain aspects, the toxin is selected from the group of toxins consisting of saporin, diphtheria toxin, pseudomonas exotoxin A, Ricin A chain derivatives, small molecule toxins and combinations thereof. In certain aspects, the toxin is a saporin. In certain embodiments, the toxin inactivates ribosomes (e.g., Shiga-like toxin chain A and bouganin, both of which are ribosome-inactivating proteins). In certain embodiments, the toxin inhibits protein synthesis. In certain aspects, the toxin is not a radioimmunotoxin. In certain embodiments, the toxin exerts its effects upon gaining entry into an intracellular compartment of one or more cells in the target tissue. In some embodiments, the methods and compositions disclosed herein do not induce cell death through DNA-damage. In some embodiments the toxin induces cell death regardless of the cell cycle stage of the cell.

In certain aspects, the toxin is selected from the group of toxins consisting of abrin toxin, modeccin toxin, gelonin toxin, momordin toxin, trichosanthin toxin, luffin toxin and combinations thereof.

In certain aspects, the toxin comprises Shiga-like toxin chain A.

In certain aspects, the toxin comprises bouganin.

In various embodiments of any aspect of the present inventions, the toxins useful in accordance with the immunotoxin compositions and methods of the present invention comprise one or more DNA-damaging molecules. For example, the selected toxin may comprise one or more anti-tubulin agents (e.g. maytansines) or tubulin inhibitors, DNA crosslinking agents, DNA alkylating agents and cell cycle or mitotic disrupters.

In certain embodiments of any aspect of the present inventions, the toxin inhibits RNA polymerase II and/or III (e.g., mammalian RNA polymerase II). In certain aspects such an RNA polymerase II and/or III inhibitor toxin is or comprises one or more amatoxins or a functional fragment, derivative or analog thereof. For example, contemplated toxins for use in accordance with any of the methods or compositions disclosed herein may include or comprise one or more amatoxins selected from the group consisting of α-amanitin, β-amanitin, γ-amanitin, £-amanitin, amanin, amaninamide, amanullin, amanullinic acid and any functional fragments, derivatives or analogs thereof.

Contemplated herein is the coupling or conjugation of an agent (e.g., an antibody) to a toxin (e.g., saporin) to facilitate the targeted delivery of such agents to cells of a target tissue. In certain aspects, the agent is directly coupled to the toxin, for example as a chimeric fusion protein. Alternatively, in certain aspects, the agent is indirectly coupled to the toxin (e.g., using a streptavidin chimera). In certain embodiments the coupling of the agent and toxin is facilitated by a streptavidin-biotin interaction (an example of an indirect linkage). In certain embodiments, the agent is biotinylated. In certain aspects, the toxin is biotinylated. In certain embodiments, the agent is coupled to a streptavidin-toxin chimera. In certain aspects, the toxin is coupled to a streptavidin-toxin chimera.

In certain aspects, the ratio of agent (e.g., antibody) to streptavidin-toxin is about 1:1, about 1:4, about 2:1 or about 4:1.

In certain aspects, the ratio of agent (e.g., antibody) to toxin is about 1:2, about 1:2.5, about 1:2.8, about 1:3, about 1:3.5, about 1:4, about 1:4.5, about 1:5, 1:6 or about 1:8.

In certain aspects, the immunotoxins disclosed herein may be prepared by conjugating a primary antibody to a secondary antibody. For example, a primary antibody that recognizes and binds to a marker (e.g., CD45) may be conjugated to a secondary antibody, which is in turn conjugated to a toxin (e.g., saporin), thereby resulting in the secondary antibody/toxin construct being “piggybacked” onto the primary antibody (e.g., a secondary antibody may recognize and bind to the heavy chain of the primary antibody). In certain embodiments, upon binding of the primary antibody to a marker, the entire immunotoxin construct comprising both the primary and secondary antibodies is internalized by cells expressing such marker. In some embodiments, internalization of such an immunotoxin construct causes cell death.

In certain aspects, the methods disclosed herein further comprise a step of administering a stem cell population to the target tissues of the subject, wherein the administered stem cell population engrafts in the target tissues of the subject. In certain embodiments, the step of administering or transplanting a stem cell population is performed after the endogenous stem cells (e.g., hematopoietic stem cells) or progenitor cells are depleted or ablated from the target tissues either partially or fully. In a preferred embodiment, such administering step is performed after the subject's target tissue (e.g., bone marrow tissue) has been conditioned in accordance with the methods and compositions disclosed herein. In some embodiments, the stem cell population is administered to the target tissues of the subject after the immunotoxin (e.g., an anti-CD45-SAP immunotoxin) has cleared or dissipated from the subject's target tissues such that the level of immunotoxin remaining in the target tissue of the subject does not induce significant cell death in the transplanted cell population. For example, in some embodiments, the stem cell population is administered to the target tissue of the subject about two to about eighteen days after the administration of the immunotoxin. In some embodiments, the stem cell population is administered to the target tissue of the subject at least one, two, three, four, five, six, seven, eight, nine, ten, twelve, twelve, thirteen, fourteen, fifteen, eighteen, twenty one, thirty six, forty two, fifty six, sixty three, seventy, eighty, ninety, one hundred, one hundred and twenty days or more, after the immunotoxin has cleared or dissipated from the target tissues of the subject.

In some embodiments, such methods disclosed herein increase the efficiency of the engraftment of the administered stem cell population in the target tissue, as compared to a method performed using only the step of administering the stem cell population to the target tissue of the subject. For example, in certain embodiments, the efficiency of engraftment is increased by at least about 5-100%, e.g., 5, 10, 15, 20, 25, 50, 75, 100% or more.

The methods and compositions disclosed herein may be used to condition a subject's tissues (e.g., bone marrow) for engraftment or transplant and following such conditioning, a stem cell population is administered to the subject's target tissues. In certain aspects, the stem cell population comprises an exogenous stem cell population. In some embodiments, the stem cell population comprises the subject's endogenous stem cells (e.g., endogenous stem cells that have been genetically modified to correct a disease or genetic defect).

In certain embodiments, the methods and compositions disclosed herein cause an increase in granulocyte colony stimulating factor (GCSF). In certain aspects, the methods and compositions disclosed herein cause an increase in macrophage colony stimulating factor (MCSF). In certain embodiments, the methods and compositions disclosed herein cause an increase in endogenous myeloid cells. Without wishing to be bound by any particular theory or mechanism of action, the increase in endogenous myeloid cells that is observed following administration of the agents, toxins and related conjugates disclosed herein may occur as a result of an increase in the subject's endogenous GCSF and/or MCSF. Accordingly, in certain embodiments, such an increase in endogenous myeloid cells occurs as a result of an increase in granulocyte colony stimulating factor (GCSF) and/or macrophage colony stimulating factor (MCSF) that may occur secondary to the methods and compositions disclosed herein. In certain aspects, the methods and compositions disclosed herein do not deplete or ablate endogenous lymphoid cells.

In certain aspects, following conditioning of a subject's target tissues in accordance with the methods and compositions disclosed herein the subject's innate immunity is preserved. In certain aspects, following conditioning of a subject's tissues in accordance with the methods and compositions disclosed herein the subject's adaptive immunity is preserved. In certain embodiments, the methods and compositions disclosed herein preserve thymic integrity of the subject. Similarly, in some embodiments, the methods and compositions disclosed herein preserve vascular integrity of the subject.

In some embodiments, conditioning of a subject's target tissues in accordance with the methods and compositions disclosed herein achieves at least about 5-90% engraftment of the exogenous stem cell population. For example, conditioning of a subject's tissues in accordance with the methods and compositions disclosed herein achieves at least about 5%, 10%, 12.5%, 15%, 17.5%, 20%, 25%, 30%, 35%, 40%, 45%, 50%, 55%, 60%, 65%, 70%, 75%, 80%, 85%, 90%, 95%, 97.5%, 99% or more engraftment of the exogenous stem cell population.

In certain embodiments, conditioning of a subject's tissues in accordance with the methods and compositions disclosed herein achieves at least about 5-90% donor chimerism (e.g., 20% donor chimerism) in the subject's target tissue (e.g., bone marrow) four months post-administration of the exogenous stem cell population to the subject. For example, in certain embodiments, conditioning of a subject's tissues in accordance with the methods and compositions disclosed herein achieves at least about 5%, 10%, 12.5%, 15%, 17.5%, 20%, 25%, 30%, 35%, 40%, 45%, 50%, 55%, 60%, 65%, 70%, 75%, 80%, 85%, 90%, 95%, 97.5%, 99% or more donor chimerism in the target tissues of the subject four months post-administration of the exogenous stem cell population to the subject.

The methods and compositions disclosed herein may be used to condition bone marrow tissue. In certain aspects, the agents (e.g., an anti-CD45-toxin conjugate) disclosed herein are useful for non-myeloablative conditioning, for example, bone marrow conditioning in advance of hematopoietic stem cell transplantation.

The methods and compositions disclosed herein may be used to treat, cure or correct a number of diseases, including, for example, a disease selected from the group consisting of sickle cell anemia, thalassemias, Fanconi anemia, Wiskott-Aldrich syndrome, adenosine deaminase SCID (ADA SCID), HIV/AIDS, metachromatic leukodystrophy, Diamond-Blackfan anemia and Schwachman-Diamond syndrome. Preferably, such methods and compositions are useful for treating such diseases without causing the toxicities that are observed in response to traditional conditioning therapies, such as irradiation.

In certain aspects, the subject has a non-malignant hemoglobinopathy (e.g., a hemoglobinopathy selected from the group consisting of sickle cell anemia, thalassemia, Fanconi anemia, and Wiskott-Aldrich syndrome). In certain aspects, the subject has an immunodeficiency. For example, in certain embodiments, the subject has a congenital immunodeficiency. Alternatively, in other aspects, the subject has an acquired immunodeficiency (e.g., an acquired immunodeficiency selected from the group consisting of HIV and AIDS). In yet other embodiments, the subject has a stem cell disorder selected from the group of disorders consisting of a non-malignant hemoglobinopathy, an immunodeficiency and cancer. In some embodiments, the subject has, suffers from or is otherwise affected by a metabolic disorder (e.g., a metabolic disorder selected from the group consisting of glycogen storage diseases, mucopolysccharidoses, Gaucher's Disease, Hurlers Disease, sphingolipidoses and metachromatic leukodystrophy). In some embodiments, the subject has, suffers from or is otherwise affected by a malignancy. In some embodiments, the subject has, suffers from or is otherwise affected by a disease or condition selected from the group consisting of severe combined immunodeficiency, Wiscott-Aldrich syndrome, hyper IGM syndrome, Chédiak-Higashi disease, hereditary lymphohistiocytosis, osteopetrosis, osteogenesis imperfect, the storage diseases, thalassemia major, sickle cell disease, systemic sclerosis, systemic lupus erythematosus, multiple sclerosis, and juvenile rheumatoid arthritis. For example, in certain embodiments the subject suffers from a malignancy selected from the group consisting of hematologic cancers (e.g., leukemia, lymphoma, multiple myeloma and myelodysplastic syndrome) and neuroblastoma.

In certain aspects, the immunotoxin compositions disclosed herein may be used to induce solid organ transplant tolerance (e.g., inducing immunogenic tolerance in connection with kidney transplant). In such embodiments, the immunotoxin compositions and methods disclosed herein may be used to deplete or ablate a population of cells from a target tissue (e.g., to deplete HSCs from the bone marrow stem cell niche). Following such depletion of cells from the target tissues, a population of stem or progenitor cells from the organ donor (e.g., HSCs from the organ donor) may be administered to the transplant recipient and following the engraftment of such stem or progenitor cells, a temporary of stable mixed chimerism achieved, thereby enabling long-term transplant organ tolerance without the need for further immunosuppressive agents.

In certain aspects, the subject is a mammal (e.g., the subject is a human). In certain aspects, the subject is immunocompetent. Alternatively, in certain embodiments, the subject is immunocompromised.

Also disclosed herein are methods of identifying a candidate agent for selectively depleting or ablating an endogenous stem cell population, such methods comprising the steps of: (a) contacting a sample comprising the stem cell population with a test agent coupled (e.g., functionally coupled) to a toxin; and (b) detecting whether one or more cells of the stem cell population are depleted or ablated from the sample; wherein the depletion or ablation of one or more cells of the stem cell population following the contacting step identifies the test agent as a candidate agent. In some embodiments, the cell is contacted with the test agent for at least about 2-24 hours.

In some embodiments, the cell is a human cell. In some embodiments, the cell is a mouse cell. In certain embodiments, the cell is a stem cell. In certain aspects, such cells comprise hematopoietic stem cells or progenitor cells. In some embodiments, the hematopoietic stem cells or progenitor cells express one or more markers selected from the group of markers consisting of HLA-DR, CD11a, CD18, CD34, CD41/61, CD43, CD45, CD49d (VLA-4), CD49f (VLA-6), CD51, CD58, CD71, CD84, CD97, CD134, CD162, CD166, CD184 (CXCR4), CD205 and CD361. In some embodiments, the human hematopoietic stem cells or progenitor cells express CD34.

In certain embodiments, the targeted cells comprise human hematopoietic stem cells expressing one or more markers that may be targeted and to which the agents that comprise the immunotoxin selectively bind, such markers selected from the group consisting of CD7, CDw12, CD13, CD15, CD19, CD21, CD22, CD29, CD30, CD33, CD34, CD36, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD99, CD104, CD105, CD109, CD111, CD112, CD114, CD115, CD123, CD124, CD126, CD127, CD130, CD131, CD135, CD138, CD151, CD157, CD162, CD164, CD168, CD172a, CD173, CD174, CD175, CD175s, CD176, CD183, CD191, CD200, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, and CD350.

In certain embodiments, the targeted cells comprise human hematopoietic stem cells expressing one or more markers that may be targeted and to which the agents that comprise the immunotoxin selectively bind, such markers selected from the group consisting of CD11a, CD18, CD37, CD47, CD52, CD58, CD62L, CD69, CD74, CD97, CD103, CD132, CD156a, CD179a, CD179b, CD184, CD232, CD244, CD252, CD302, CD305, CD317, and CD361.

In certain embodiments, the targeted cells comprise human hematopoietic stem cells expressing one or more markers that may be targeted and to which the agents that comprise the immunotoxin selectively bind, such markers being selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L. In certain embodiments, the targeted cells comprise human hematopoietic stem cells expressing one or more markers that may be targeted and to which the agents that comprise the immunotoxin selectively bind, such markers selected from the group consisting of CD51/61, CD72, CD45RA, CD107a, CD45RB, CD7, CD13, CD132 and CD321.

In certain embodiments, the test agent is an antibody. In certain aspects, the test agent is a ligand. In some embodiments, the toxin is internalized by the one or more cells of the HSC or progenitor cell population. In some embodiments, the internalization comprises receptor-mediated internalization. In certain embodiments, the toxin is selected from the group of toxins consisting of saporin, diphtheria toxin, pseudomonas exotoxin A, Ricin A chain derivatives, a small molecule toxin and combinations thereof. In certain aspects, the toxin is selected from the group of toxins consisting of abrin toxin, modeccin toxin, gelonin toxin, momordin toxin, trichosanthin toxin, luffin toxin and combinations thereof. In some embodiments, the toxin is or comprises an amatoxin (e.g., α-amanitin).

While certain embodiments disclosed herein contemplate the use of, for example, an agent-toxin conjugate to deplete or condition a tissue (e.g., bone marrow tissue), or to receptor-mediated internalization of a toxin, the inventions disclosed herein are not limited to such embodiments. Rather, contemplated herein are any methods that may be used to selectively deliver a toxin intracellularly to the cells of a target tissue. For example, in certain embodiments, disclosed herein are methods of delivering toxins intracellularly using pore-mediated internalization.

In certain embodiments, disclosed herein are methods of conditioning a subject for engraftment, such methods comprising selectively depleting or ablating an endogenous stem cell population in a target tissue (e.g., bone marrow tissue) of the subject by: (a) administering to the subject an effective amount of a pore-forming chimera comprising a mutant protective antigen (mut-PA) coupled (e.g., functionally coupled) to an agent, and thereby forming one or more pores in the cell membrane of the endogenous stem cell population; and (b) administering to the subject an effective amount of a second chimera, wherein the second chimera comprises a factor (e.g., an enzymatic factor) coupled to a toxin, wherein the factor is selected from the group consisting of lethal factor N-terminus (LFN), edema factor N-terminus (EFN) or fragments thereof, and wherein the toxin is internalized by the endogenous stem cell population, thereby selectively depleting or ablating the endogenous stem cell population in the target tissue and conditioning the subject for engraftment.

In certain embodiments, the present inventions are directed to methods of engrafting stem cells in a subject, such methods comprising the steps of: (a) administering to the subject an effective amount (e.g., 1.5 mg/kg) of a pore-forming chimera comprising a mutant protective antigen (mut-PA) coupled to an agent, and thereby forming one or more pores in the cell membrane of the endogenous stem cell population; (b) administering to the subject an effective amount of a second chimera, wherein the second chimera comprises a factor (e.g., an enzymatic factor) coupled to a toxin, wherein the factor is selected from the group consisting of lethal factor N-terminus (LFN), edema factor N-terminus (EFN) or fragments thereof, and wherein the toxin is internalized by the endogenous stem cell population, thereby depleting or ablating the endogenous stem cell population in the target tissue (e.g., bone marrow tissue); and (c) administering a stem cell population to the target tissue of the subject, wherein the administered stem cell population engrafts in the target tissue of the subject. In some embodiments, the stem cell population is administered to the target tissues of the subject after the toxin (e.g., a diphtheria toxin A chain chimera fusion to LFN (LFN-DTA)) has cleared or dissipated from the subject's target tissues.

In some embodiments, the agent is selected from the group consisting of a scfv, a Fab, a discfv, a biscFv, a tri-scfv, a tandem scfv, an aptamer, an antibody and a ligand. In certain embodiments, the agent is a single-chain variable fragment (scFv). In certain aspects, the agent is a bispecific antibody.

In still other embodiments, the agent is a ligand. For example, such a ligand may be selected from the group of ligands consisting of stem cell factor (SCF), CXCL12: Stromal derived factor 1 (SDF1), Angiopoietin 1 to 4 (Ang1, Ang2, Ang3, Ang4), TPO (thrombopoietin), Erythropoietin, FLT3L, VLA4, VLA6, IL-1, IL-3, IL-6, IL-18, G-CSF, Oncostatin M, LIF and combinations thereof.

In certain embodiments of the methods disclosed herein, the toxin is internalized by a pore-mediated internalization. In certain embodiments, the toxin is saporin. In certain embodiments, the toxin inactivates ribosomes (e.g., one or more of the ribosome-inactivating toxins Shiga-like toxin chain A and bouganin). In certain embodiments, the toxin inhibits protein synthesis. In certain aspects, the toxin is selected from the group of toxins consisting of saporin, diphtheria toxin, pseudomonas exotoxin A, Ricin A chain derivatives, small molecule toxins and combinations thereof. In some embodiments, the toxin is or comprises an amatoxin (e.g., α-amanitin). In some embodiments, the toxin is selected from the group consisting of abrin toxin, modeccin toxin, gelonin toxin, momordin toxin, trichosanthin toxin, luffin toxin and combinations thereof.

In certain aspects, the toxin comprises Shiga-like toxin chain A.

In certain aspects, the toxin comprises bouganin.

In certain embodiments, the endogenous stem cell population comprises hematopoietic stem cells. In certain embodiments, the hematopoietic stem cells or progenitor cells comprise or express one or more markers. For example, in certain embodiments the hematopoietic stem cells or progenitor cells express one or more markers selected from the group of markers consisting of: CD13, CD33, CD34, CD44, CD45, CD49d: VLA-4, CD49f: VLA-6, CD59, CD84, CD93, CD105: Endoglin, CD123: IL-3R, CD126: IL-6R, CD135: Flt3 receptor, CD166: ALCAM, CD184: CXCR4, Prominin 2, Erythropoietin R, CD244, Tie1, Tie2, G-CSFR or CSF3R, IL-1R, gp130, Leukemia inhibitory factor Receptor, oncostatin M receptor, Embigin and IL-18R. In certain embodiments, the hematopoietic stem cells or progenitor cells express one or more markers selected from the group consisting of HLA-DR, CD11a, CD18, CD34, CD41/61, CD43, CD45, CD47, CD58, CD71, CD84, CD97, CD162, CD166, CD205 and CD361. In certain aspects, the hematopoietic stem cells or progenitor cells express one or more markers selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L. In certain aspects, the hematopoietic stem cells or progenitor cells express one or more markers selected from the group consisting of CD51/61, CD72, CD45RA, CD107a, CD45RB, CD7, CD13, CD132 and CD321. In certain aspects, the agent selectively binds to the marker. In certain aspects, upon binding of the agent to the marker, the immunotoxin is internalized by the cells expressing such marker.

In certain embodiments, the subject is a mammal. In certain embodiments, the mammal is a human. In certain embodiments, the methods and compositions disclosed herein may be used to treat, cure or otherwise ameliorate a disease or condition in a subject affected thereby. Accordingly, in certain aspects, the subject has a non-malignant hemoglobinopathy. For example, such a subject may be affected by a hemoglobinopathy selected from the group consisting of sickle cell anemia, thalassemia, Fanconi anemia, and Wiskott-Aldrich syndrome.

In certain aspects, the subject has an immunodeficiency. For example, in certain embodiments, the immunodeficiency is a congenital immunodeficiency. Alternatively, in certain aspects the immunodeficiency is an acquired immunodeficiency. For example, an acquired immunodeficiency selected from the group consisting of HIV and AIDS.

In still other embodiments, the subject has or is otherwise affected by the stem cell disorder selected from the group of disorders consisting of a non-malignant hemoglobinopathy, an immunodeficiency and cancer.

In various embodiments of any aspect of the present inventions, the compositions and methods disclosed herein further comprise administering to the subject one or more mobilizing agents (e.g., a combination of a CXCR2 agonist and a CXCR4 antagonist). For example, the compositions disclosed herein may be co-administered with one or more mobilizing agents and/or may be administered subsequent to the administration of the one or more mobilizing agents (e.g., 15 minutes post-administration of the mobilizing agent). In certain aspects, the mobilizing agent is or comprises filgrastim (GCSF). In certain aspects, the mobilizing agent is selected from the group consisting of a CXCR2 agonist (e.g., Gro-beta), a CXCR4 antagonist (e.g., plerixafor), and combinations thereof. In certain embodiments, the mobilizing agent comprises Gro-beta. In certain aspects, the mobilizing agent comprises Gro-betaΔ4. In certain embodiments, the mobilizing agent comprises plerixafor. In certain aspects, the mobilizing agents comprise Gro-beta and plerixafor. In certain aspects, the mobilizing agents comprise Gro-betaΔ4 and plerixafor. In certain aspects, the mobilizing agent comprises a heparan sulfate inhibitor.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates the results of an immunotoxin screening assay against KG1a hematopoietic progenitor cells. KG1a hematopoietic progenitor cells were incubated with a 3 nM or 10 nM concentration of the primary antibody with secondary antibody-saporin conjugate at a concentration of 20 nM. Cells were incubated for 72 hours and cell death was assessed by the MTS assay, which measured metabolic activity. As a 100% death control, cells were incubated with 10 μM staurosporine.

FIG. 2 illustrates the results of an immunotoxin screening assay against primary human bone marrow CD34+ cells with a 3 nM or 10 nM concentration of the primary antibody with secondary antibody-saporin conjugate at a concentration of 20 nM. Cells were incubated for 120 hours and cell death was assessed by the MTS assay, which measured metabolic activity. As a 100% death control, cells were incubated with 10 μM staurosporine.

### DETAILED DESCRIPTION OF THE INVENTION

The compositions and methods disclosed herein generally relate to compositions, methods, therapies and regimens that are useful for conditioning a subject's tissues for engraftment or transplant (e.g., hematopoietic stem cell transplant). In particular, such compositions and methods selectively target a marker (e.g., a cell surface marker such as the CD45 receptor) and facilitate the intracellular delivery of an immunotoxin to one or more cells (e.g., CD45+ cells) of the target tissue, for example, hematopoietic stem cells (HSCs) and/or progenitor cells in the bone marrow tissue of a subject. By selectively targeting cells expressing a selected marker (e.g., CD45), the compositions and methods disclosed herein are able to exert their cytotoxic effect on those targeted cells, while sparing, minimizing, and in certain instances eliminating, adverse effects on non-targeted cells and tissues. For example, in certain instances, the compositions and methods disclosed herein selectively ablate or deplete the endogenous stem cell niche of a target tissue (e.g., bone marrow tissue); however, in contrast to traditional conditioning regimens (e.g., the reduced conditioning regimen for sickle cell anemia disclosed by Bolanos-Meade, et al., Blood (2012), 120(22): 4286), such compositions and methods do not induce life-threatening neutropenia, thrombocytopenia and/or anemia in the subject.

In certain aspects, the compositions and methods disclosed herein relate to the targeting, ablation and/or depletion of hematopoietic stem or progenitor cells (HSPCs) residing in the target tissues of a subject, for example, hematopoietic stem or progenitor cells within a stem cell niche (e.g., a subject's bone marrow). As used herein, “hematopoietic stem cells” refers to stem cells that can differentiate into the hematopoietic lineage and give rise to all blood cell types such as white blood cells and red blood cells, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells). Stem cells are defined by their ability to form multiple cell types (multipotency) and their ability to self-renew. Human hematopoietic stem cells can be identified, for example by cell surface markers such as CD34+, CD90+, CD49f+, CD38− and CD45RA−. Murine hematopoietic stem cells can be identified, for example by cell surface markers such as CD34−, CD133+, CD48−, CD150+, CD244−, cKit+, Scal+, and lack of lineage markers (negative for B220, CD3, CD4, CD8, Mac1, Gr1, and Ter119, among others). The compositions and methods described herein may be useful for the depletion or ablation any stem cell, including, but not limited to, peripheral blood stem cells, bone marrow stem cells, umbilical cord stem cells, genetically modified stem cells, etc.

As used herein, the term “hematopoietic progenitor cells” encompasses pluripotent cells which are committed to the hematopoietic cell lineage, generally do not self-renew, and are capable of differentiating into several cell types of the hematopoietic system, such as granulocytes, monocytes, erythrocytes, megakaryocytes, B-cells and T-cells, including, but not limited to, short term hematopoietic stem cells (ST-HSCs), multi-potent progenitor cells (MPPs), common myeloid progenitor cells (CMPs), granulocyte-monocyte progenitor cells (GMPs), megakaryocyte-erythrocyte progenitor cells (MEPs), and committed lymphoid progenitor cells (CLPs). The presence of hematopoietic progenitor cells can be determined functionally as colony forming unit cells (CFU-Cs) in complete methylcellulose assays, or phenotypically through the detection of cell surface markers (e.g., CD45, CD34+, Ter119−, CD16/32, CD127, cKit, Scal) using assays known to those of skill in the art.

The present inventions contemplate ablating or depleting hematopoietic stem cells and/or progenitor cells for any purpose which would be desirable to the skilled artisan. In some embodiments, the hematopoietic stem cells and/or progenitor cells are ablated or depleted from the target tissues of a subject (e.g., the stem cell niche) to condition the subject for engraftment of transplanted hematopoietic stem cells and/or progenitors cells, for example by decreasing the number of or eliminating hematopoietic stem cells and/or progenitor cells in a stem cell niche (e.g., bone marrow) into which the transplanted cells can engraft.

While certain aspects of the present invention contemplate the ablation or depletion of, for example, hematopoietic stem cells from the stem cell niche, the present inventions may also be useful for ablating or depleting non-hematopoietic stem cells that are involved in maintaining the stem cell niche. For example, the compounds and methods disclosed herein may be used to target non-HSC, hematopoietic subsets that play a role in niche maintenance of hematopoietic stem cells. Such hematopoietic subsets that may be targeted, ablated or depleted using the compositions and methods disclosed herein include, for example, T-cells expressing CD4, CD3 or CD8; B-cells expressing B220 or CD19; and myeloid cells expressing Gr-1 or Mac-1 (CD11b).

As used herein the terms “ablate” and “ablation” generally refer to the partial or complete removal of a population of cells (e.g., hematopoietic stem cells or progenitor cells) from the target tissues (e.g., bone marrow tissues of a subject). In certain aspects, such ablation comprises a complete removal or depletion of such cells from the target tissue. Alternatively, in other aspects, such ablation is a partial removal or depletion of such cells (e.g., HSCs or progenitor cells) from the target tissue. For example, in certain aspects, the methods and compositions disclosed herein result in at least about 5%, 10%, 12.5%, 15%, 20%, 25%, 30%, 35%, 40%, 45%, 50%, 55%, 60%, 65%, 70%, 75%, 80%, 85%, 90%, 92.5%, 95%, 97.5%, 98% or 99% depletion of the cells (e.g., HSCs or progenitor cells) of the target tissue.

The CD45 receptor is a unique and ubiquitous membrane glycoprotein that is expressed on almost all hematopoietic cells. The inventions disclosed herein are based in-part upon the discovery that certain markers (e.g., cell surface markers such as CD45) have internalizing properties that may be exploited to facilitate the intracellular delivery of a toxin (e.g., a toxin such as saporin) to the cells of a target tissue and thereby induce cell death. Accordingly, in certain embodiments the agents (e.g., antibodies and/or ligands) and compositions disclosed herein are characterized as being internalizing and thus can cause or otherwise facilitate the intracellular delivery of one or more immunotoxins to cells of the target tissue that express a targeted marker (e.g., a targeted cell surface marker).

In certain aspects, the inventions disclosed herein contemplate the selection of one or more markers (e.g., a cell surface marker) to facilitate the selective targeting of the agents to the cells of a target tissue. As used herein, the term “selectively” means that the agent (e.g., an antibody) preferentially or discriminatorily recognizes and/or binds to a marker or a fragment or epitope of such marker (e.g., a cell surface marker). Exemplary antibody agents that selectively recognize and/or bind a cell surface marker (e.g., CD45 and CD34) and that may be used in accordance with the present inventions include, clone 104, clone 30F11, clone 3C11, clone MEM-28, clone HI30, clone 581 and clone 4H11. In certain aspects, the agent comprises an antibody that selectively recognizes and/or binds to the CD34 marker (e.g., clone 581 or clone 4H11). In certain aspects, the agent comprises an antibody that selectively recognizes and/or binds to the CD45 marker (e.g., clone MEM-28 or clone HI30). In certain aspects, the agent is an antibody selected from the group consisting of clone L243, clone TS2/4, clone TS1/18, clone 581, clone 4H11, clone A2A9/6, clone CD43-10G7, clone BHPT-1, clone orb12060, clone 2D1, clone CC2C6, clone TS2/9, clone CY1G4, clone OKT9, clone CD84.1.21, clone VIM3b, clone A3C6E2, clone EMK08, clone TMP4, clone KPL-1, clone 3a6, clone HD83 and clone MEM-216. By selectively targeting the cells of the target tissues, the methods and compositions disclosed herein may reduce, limit or otherwise avoid toxicities that have historically plagued traditional conditioning regimens and that result in life-threatening complications.

As used herein, the term “marker” generally refers to any protein, receptor, antigen, carbohydrates, lipids or other moieties that may be located or expressed on the surface of the cells of the target tissue and that can be used to discriminate a cell population. In particular, such markers may be used to selectively target the agents that comprise the immunotoxin compositions disclosed herein to the cells of the target tissue. While certain embodiments disclosed herein contemplate the selective targeting of a cell using, for example the CD34 and/or CD45 markers, the inventions are not limited to those markers. Rather, the present inventions contemplate the selection and use of any markers (e.g., cell surface markers) that may be useful or suitable for selectively targeting a cell population, inclusive of any yet to be discovered markers. Preferably, the selected marker is selectively expressed on the surface of the target cell population, thereby facilitating the selective or discriminatory targeting of such cell population using the agents (e.g., antibodies and/or ligands) disclosed herein. For example, in certain aspects, the selected marker is expressed on hematopoietic stem cells or progenitor cells. Exemplary markers may be selected from the group of markers consisting of HLA-DR, CD11a, CD18, CD34, CD41/61, CD43, CD45, CD49d (VLA-4), CD49f (VLA-6), CD51, CD58, CD71, CD84, CD97, CD134, CD162, CD166, CD184 (CXCR4), CD205 and CD361. In certain aspects, the marker is selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L. In certain aspects, the marker is selected from the group consisting of CD51/61, CD72, CD45RA, CD107a, CD45RB, CD7, CD13, CD132 and CD321. In certain embodiments, the selected marker is only expressed on the targeted cell population (e.g., the target HSC population), thereby limiting or avoiding the “off-target” effects that have limited the utility of traditional conditioning regimens.

In certain embodiments, the selection of a marker may be made based upon comparing the detected expression of such a marker (e.g., a cell surface marker) on a target cell relative the expression of such marker on a control population of cells. For example, the expression of a marker on a HSC or progenitor cell can be compared to the mean expression of the same marker on other cells.

In certain embodiments, the marker is a receptor. Exemplary human receptors that may be used or selected as markers in accordance with the inventions disclosed herein may be selected from the group of markers consisting of CD13, CD33, CD34, CD44, CD45, CD49d: VLA-4, CD49f: VLA-6, CD59, CD84, CD93, CD105: Endoglin, CD123: IL-3R, CD126: IL-6R, CD135: Flt3 receptor, CD166: ALCAM, CD184: CXCR4, Prominin 2, Erythropoietin R, CD244, Tie1, Tie2, G-CSFR or CSF3R, IL-1R, gp130, Leukemia inhibitory factor Receptor, oncostatin M receptor, Embigin and IL-18R.

In certain aspects, exemplary markers that are expressed on human hematopoietic stem cells, that may be targeted and to which the agents that comprise the immunotoxin selectively bind may be selected from the group consisting of CD7, CDw12, CD13, CD15, CD19, CD21, CD22, CD29, CD30, CD33, CD34, CD36, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD99, CD104, CD105, CD109, CD111, CD112, CD114, CD115, CD123, CD124, CD126, CD127, CD130, CD131, CD135, CD138, CD151, CD157, CD162, CD164, CD168, CD172a, CD173, CD174, CD175, CD175s, CD176, CD183, CD191, CD200, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, and CD350.

In some embodiments, exemplary markers that are expressed on human hematopoietic stem cells, that may be targets and to which the agents that comprise the immunotoxin selectively bind may be selected from the group consisting of CD11a, CD18, CD37, CD47, CD52, CD58, CD62L, CD69, CD74, CD97, CD103, CD132, CD156a, CD179a, CD179b, CD184, CD232, CD244, CD252, CD302, CD305, CD317, and CD361. In certain aspects, the marker is selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L. In yet other aspects, the marker is selected from the group consisting of CD51/61, CD72, CD45RA, CD107a, CD45RB, CD7, CD13, CD132 and CD321.

Exemplary mouse receptors that may be used of selected as markers in accordance with the inventions disclosed herein may include, for example, Sca-1.

Exemplary ligands that may be used or selected as markers in accordance with the inventions disclosed herein may be selected from the group of markers consisting of CXCL12: Stromal derived factor 1 (SDF1), Angiopoietin 1 to 4 (Ang1, Ang2, Ang3, Ang4), TPO (thrombopoietin), Erythropoietin, FLT3L, VLA-4, VLA-6, IL-1, IL-3, IL-6, IL-18, G-CSF, Oncostatin M and LIF.

The compositions disclosed herein comprise an agent to facilitating targeting of such composition to, for example, an endogenous hematopoietic stem cell or progenitor cell population in a target tissue of a subject. As used herein, the term “agent” refers to any substance, molecule, compound or moiety, such as an antibody or a ligand or an aptamer, that may be used for, or that otherwise facilitates the targeting or directing of a moiety, such as a toxin coupled to such agent, to one or more cells (e.g., one or more hematopoietic stem cells or progenitor cells in the target tissue of a subject). In certain aspects, the agent selectively targets the cells in a target tissue (e.g., bone marrow tissue), causing the moiety (e.g., a toxin) coupled thereto to be internalized by such cells and thereby ablate or deplete such cells from the target tissue. In certain embodiments, the agent selectively recognizes and/or binds to a marker or to a fragment or epitope of such marker (e.g., a cell surface marker, such as a receptor).

The agents disclosed herein include, without limitation, any agents that can selectively target, bind to or recognize a marker or epitope that may be differentially expressed on the cell surface of the cells of the target tissue. In some embodiments, such agents direct or target the immunotoxins disclosed herein to the cells of the target tissue (e.g., cancer stem cells), thereby depleting or ablating such cells from the target tissue and conditioning such target tissue. In some embodiments, the agent is or comprises a ligand. In some embodiments, the agent is or comprises an aptamer. The agents of the present invention are not limited to the foregoing illustrative examples; rather any agent that can selectively target, bind to or recognize a marker or epitope expressed on the cell surface of the cells of target tissues may be used. In certain embodiments, the agent is recombinantly prepared.

In certain aspects, the agent is or comprises an antibody (e.g., a monoclonal or polyclonal antibody). The antibodies of the present invention can be polyclonal or monoclonal, and the term “antibody” is intended to encompass both polyclonal and monoclonal antibodies. For example, in certain aspects the antibody is selected from the group consisting of clone 104, clone 30F11, clone 3C11, clone MEM-28, clone HI30, clone 581 and clone 4H11. In certain embodiments, the agent is an antibody comprising a complementarity determining region that is the same as the complementarity determining region for one or more antibodies selected from the group consisting of clone 104, clone 30F11, clone 3C11, clone MEM-28, clone HI30, clone 581 and clone 4H11. In certain embodiments, the agent is an antibody that binds to the same epitope as one or more antibodies selected from the group consisting of 104, clone 30F11, clone 3C11, clone MEM-28, clone HI30, clone 581 and clone 4H11.

In certain aspects the antibody is selected from the group consisting of clone L243, clone TS2/4, clone TS1/18, clone 581, clone 4H11, clone A2A9/6, clone CD43-10G7, clone BHPT-1, clone orb12060, clone 2D1, clone CC2C6, clone TS2/9, clone CY1G4, clone OKT9, clone CD84.1.21, clone VIM3b, clone A3C6E2, clone EMK08, clone TMP4, clone KPL-1, clone 3a6, clone HD83 and clone MEM-216. In certain embodiments, the agent is an antibody comprising a complementarity determining region that is the same as the complementarity determining region for one or more antibodies selected from the group consisting of L243, clone TS2/4, clone TS1/18, clone 581, clone 4H11, clone A2A9/6, clone CD43-10G7, clone BHPT-1, clone orb12060, clone 2D1, clone CC2C6, clone TS2/9, clone CY1G4, clone OKT9, clone CD84.1.21, clone VIM3b, clone A3C6E2, clone EMK08, clone TMP4, clone KPL-1, clone 3a6, clone HD83 and clone MEM-216. In certain embodiments, the agent is an antibody that binds to the same epitope as one or more antibodies selected from the group consisting of L243, clone TS2/4, clone TS1/18, clone 581, clone 4H11, clone A2A9/6, clone CD43-10G7, clone BHPT-1, clone orb12060, clone 2D1, clone CC2C6, clone TS2/9, clone CY1G4, clone OKT9, clone CD84.1.21, clone VIM3b, clone A3C6E2, clone EMK08, clone TMP4, clone KPL-1, clone 3a6, clone HD83 and clone MEM-216. Furthermore, it is understood that the methods described herein which utilize antibodies as the agent to facilitate delivery of the immunotoxin to the cells of the target tissue can also utilize functional fragments (e.g., antigen-binding fragments) of such antibodies.

In certain embodiments, the agent comprises an antibody selected from the group consisting of clone 23C6, clone J4-117, clone HI100, clone H4A3, clone MT4, clone M-T701, clone WM15, clone TUGh4 and clone M.AB.F11.

In certain aspects, the agent comprises an antibody selected from the group consisting of clone TU39, clone TU99, clone N6B6, clone TU41, clone UM7F8, clone H5C6, clone G44-26, clone G46-2.6, clone HECA-452, clone CBR-1C2/2.1, clone 1C3, clone EBA-1, clone HIM6, clone p282 (H19), clone AK-4, clone CSLEX1, clone G28-8, clone 11G7, clone VC5, clone 28D4, clone 3A6, clone 2D7/CCR5, clone SN2, clone TU169, clone WM59, clone GHI/75, clone 9F5, clone HIP2, clone FN50, clone KPL-1, clone 1G10, clone M-A712, clone B6H12, clone VIM3b, clone MG38, clone G46-6 (L243), clone 581, clone 9F10, clone 12G5, clone 2G7, clone TU145, clone G43-25B and clone Dreg 56.

In some embodiments, agent comprises an antibody, and wherein the antibody comprises a complementarity determining region that is the same as the complementarity determining region for one or more antibodies selected from the group consisting of clone 23C6, clone J4-117, clone HI100, clone H4A3, clone MT4, clone M-T701, clone WM15, clone TUGh4 and clone M.AB.F11.

In certain aspects of the present inventions, the agent comprises an antibody, and wherein the antibody comprises a complementarity determining region that is the same as the complementarity determining region for one or more antibodies selected from the group consisting of clone TU39, clone TU99, clone N6B6, clone TU41, clone UM7F8, clone H5C6, clone G44-26, clone G46-2.6, clone HECA-452, clone CBR-1C2/2.1, clone 1C3, clone EBA-1, clone HIM6, clone p282 (H19), clone AK-4, clone CSLEX1, clone G28-8, clone 11G7, clone VC5, clone 28D4, clone 3A6, clone 2D7/CCR5, clone SN2, clone TU169, clone WM59, clone GHI/75, clone 9F5, clone HIP2, clone FN50, clone KPL-1, clone 1G10, clone M-A712, clone B6H12, clone VIM3b, clone MG38, clone G46-6 (L243), clone 581, clone 9F10, clone 12G5, clone 2G7, clone TU145, clone G43-25B and clone Dreg 56.

Antibodies of the present invention can be raised against an appropriate marker or antigen, such as, for example, isolated and/or recombinant mammalian CD34 or CD45 receptor or portions or epitopes thereof. Antibodies can be raised against a selected marker (e.g., a cell surface marker) or antigen by methods known to those skilled in the art. Such methods for raising polyclonal antibodies are well known in the art and are described in detail, for example, in Harlow et al., 1988 in: Antibodies, A Laboratory Manual, Cold Spring Harbor, N.Y.

Typically, such antibodies are raised by immunizing an animal (e.g. a rabbit, rat, mouse, donkey, etc.) by multiple subcutaneous or intraperitoneal injections of the relevant antigen (e.g., CD34 or CD45) optionally conjugated to keyhole limpet hemocyanin (KLH), serum albumin, other immunogenic carrier, diluted in sterile saline and combined with an adjuvant (e.g. Complete or Incomplete Freund's Adjuvant) to form a stable emulsion. The polyclonal antibody is then recovered from blood or ascites of the immunized animal. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc. Polyclonal antiserum can also be rendered monospecific using standard procedures (see, e.g., Agaton et al., “Selective Enrichment of Monospecific Polyclonal Antibodies for Antibody-Based Proteomics Efforts,” J Chromatography A 1043(1):33-40 (2004), which is hereby incorporated by reference in its entirety).

In some embodiments, monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety. Using the hybridoma method, a mouse, hamster, or other appropriate host animal, is immunized to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against for example, a cell surface marker such as CD34 or CD45, as determined by immunoprecipitation, immunoblotting, or by an in vitro binding assay such as radioimmunoassay (RIA) or enzyme-linked immunosorbent assay (ELISA) can then be propagated either in vitro culture using standard methods (James Goding, Monoclonal Antibodies: Principles and Practice (1986) which is hereby incorporated by reference in its entirety) or in vivo as ascites tumors in an animal. The monoclonal antibodies can then be purified from the culture medium or ascites fluid as described for polyclonal antibodies above.

In some embodiments, monoclonal antibodies can be made using recombinant DNA methods as described in U.S. Pat. No. 4,816,567 to Cabilly et al., which is hereby incorporated by reference in its entirety. The polynucleotides encoding a monoclonal antibody are isolated, such as from mature B-cells or hybridoma cells, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, and monoclonal antibodies are generated by the host cells. Recombinant monoclonal antibodies or fragments thereof of the desired species can also be isolated from phage display libraries as described (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotides encoding a monoclonal antibody can further be modified in a number of different ways using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

In some embodiments, the monoclonal antibody against a cell surface marker or antigen, such as CD34 or CD45, is a humanized antibody. In certain embodiments, the monoclonal antibody against a cell surface marker or antigen, such as HLA-DR, CD11a, CD18, CD34, CD41/61, CD43, CD45, CD47, CD58, CD71, CD84, CD97, CD162, CD166, CD205 and/or CD361, is a humanized antibody. Humanized antibodies are antibodies that contain minimal sequences from non-human (e.g. murine) antibodies within the variable regions. Such antibodies are used therapeutically to reduce antigenicity and human anti-mouse antibody responses when administered to a human subject. In practice, humanized antibodies are typically human antibodies with minimum to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the complementarity determining region (CDR) of a human antibody with that of a non-human antibody (e.g. mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Human antibodies can be directly prepared using various techniques known in the art. Immortalized human B lymphocytes immunized in vitro or isolated from an immunized individual that produces an antibody directed against a target antigen can be generated (see, e.g. Reisfeld et al., Monoclonal Antibodies and Cancer Therapy 77 (Alan R. Liss 1985) and U.S. Pat. No. 5,750,373 to Garrard, which are hereby incorporated by reference in their entirety). Also, the human antibody can be selected from a phage library, where that phage library expresses human antibodies (Vaughan et al., “Human Antibodies with Sub-Nanomolar Affinities Isolated from a Large Non-immunized Phage Display Library,” Nature Biotechnology, 14:309-314 (1996); Sheets et al., “Efficient Construction of a Large Nonimmune Phage Antibody Library: The Production of High-Affinity Human Single-Chain Antibodies to Protein Antigens,” Proc Nat'l Acad Sci USA 95:6157-6162 (1998); Hoogenboom et al., “By-passing Immunisation. Human Antibodies From Synthetic Repertoires of Germline VH Gene Segments Rearranged In Vitro,” J Mol. Biol, 227:381-8 (1992); Marks et al., “By-passing Immunization. Human Antibodies from V-gene Libraries Displayed on Phage,” J. Mol. Biol, 222:581-97 (1991), which are hereby incorporated by reference in their entirety). Humanized antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and U.S. Pat. No. 5,661,016 to Lonberg et al., which are hereby incorporated by reference in their entirety.

In some embodiments, the agents that comprise the immunotoxin compositions of the present invention include bispecific antibodies that specifically recognize one or more cell surface markers. Bispecific antibodies are antibodies that are capable of specifically recognizing and binding at least two different epitopes. Bispecific antibodies can be intact antibodies or antibody fragments. Techniques for making bispecific antibodies are common in the art (Brennan et al., “Preparation of Bispecific Antibodies by Chemical Recombination of Monoclonal Immunoglobulin G1 Fragments,” Science 229:81-3 (1985); Suresh et al., “Bispecific Monoclonal Antibodies From Hybrid Hybridomas,” Methods in Enzymol. 121:210-28 (1986); Traunecker et al., “Bispecific Single Chain Molecules (Janusins) Target Cytotoxic Lymphocytes on HIV Infected Cells,” EMBO J. 10:3655-3659 (1991); Shalaby et al., “Development of Humanized Bispecific Antibodies Reactive with Cytotoxic Lymphocytes and Tumor Cells Overexpressing the HER2 Protooncogene,” J. Exp. Med. 175:217-225 (1992); Kostelny et al., “Formation of a Bispecific Antibody by the Use of Leucine Zippers,” J. Immunol. 148: 1547-1553 (1992); Gruber et al., “Efficient Tumor Cell Lysis Mediated by a Bispecific Single Chain Antibody Expressed in Escherichia coli,” J. Immunol. 152:5368-74 (1994); and U.S. Pat. No. 5,731,168 to Carter et al., which are hereby incorporated by reference in their entirety).

In some embodiments, the use of such bispecific antibodies may facilitate the targeting of the immunotoxin compositions disclosed herein to a first cell surface marker expressed by cells of the target tissues, as well as a second marker capable of facilitating the internalization of such immunotoxin composition. Similarly, such bispecific antibodies may be used to increase the targeting precision of the immunotoxin compositions disclosed herein. In some aspects, bispecific antibodies may be useful for binding a cell surface marker of a particular cell (e.g., myeloid cells), while a second cell surface marker may also be targeted to internalize the immunotoxin composition. For example, in certain embodiments, the bispecific antibodies disclosed herein bind a cell surface marker having internalizing properties that may be exploited to facilitate the intracellular delivery of a toxin (e.g., a toxin such as saporin) to the cells of a target tissue and thereby induce cell death.

Bispecific antibodies that bind, for example, both CD34 and CD45, may be prepared by any technique known in the art. For example, in certain aspects the bispecific antibodies disclosed herein may be prepared using chemical linkage. Alternatively, such bispecific antibodies can be prepared recombinantly using a co-expression of two immunoglobulin heavy chain/light chain pairs. In some aspects, bispecific antibodies may be prepared by disulfide exchange, production of hybrid-hybridomas, by transcription and translation to produce a single polypeptide chain embodying a bispecific antibody, or transcription and translation to produce more than one polypeptide chain that can associate covalently to produce a bispecific antibody.

In some embodiments, the bispecific agents or antibodies disclosed herein binds to one or more markers selected from the group consisting of CD13, CD33, CD34, CD44, CD45, CD49d: VLA-4, CD49f: VLA-6, CD59, CD84, CD93, CD105: Endoglin, CD123: IL-3R, CD126: IL-6R, CD135: Flt3 receptor, CD166: ALCAM, CD184: CXCR4, Prominin 2, Erythropoietin R, CD244, Tie1, Tie2, G-CSFR or CSF3R, IL-1R, gp130, Leukemia inhibitory factor Receptor, oncostatin M receptor, Embigin and IL-18R. In certain embodiments, the bispecific agent or antibody disclosed herein binds to one or more markers selected from the group consisting of HLA-DR, CD11a, CD18, CD34, CD41/61, CD43, CD45, CD47, CD58, CD71, CD84, CD97, CD162, CD166, CD205 and CD361.

In some embodiments, the bispecific agents or antibodies disclosed herein bind to two or more markers selected from the group consisting of CD13, CD33, CD34, CD44, CD45, CD49d: VLA-4, CD49f: VLA-6, CD59, CD84, CD93, CD105: Endoglin, CD123: IL-3R, CD126: IL-6R, CD135: Flt3 receptor, CD166: ALCAM, CD184: CXCR4, Prominin 2, Erythropoietin R, CD244, Tie1, Tie2, G-CSFR or CSF3R, IL-1R, gp130, Leukemia inhibitory factor Receptor, oncostatin M receptor, Embigin and IL-18R. In certain embodiments, the bispecific agent or antibody disclosed herein binds to two or more markers selected from the group consisting of HLA-DR, CD11a, CD18, CD34, CD41/61, CD43, CD45, CD47, CD58, CD71, CD84, CD97, CD162, CD166, CD205 and CD361. In certain aspects, the bispecific agent or antibody disclosed herein binds to two or more markers selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L. In certain aspects, the bispecific agent or antibody disclosed herein binds to two or more markers selected from the group consisting CD51/61, CD72, CD45RA, CD107a, CD45RB, CD7, CD13, CD132 and CD321.

In certain embodiments, the bispecific agent or antibody disclosed herein binds to two or more markers expressed on human hematopoietic stem cells and selected from the group consisting of CD7, CDw12, CD13, CD15, CD19, CD21, CD22, CD29, CD30, CD33, CD34, CD36, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD99, CD104, CD105, CD109, CD111, CD112, CD114, CD115, CD123, CD124, CD126, CD127, CD130, CD131, CD135, CD138, CD151, CD157, CD162, CD164, CD168, CD172a, CD173, CD174, CD175, CD175s, CD176, CD183, CD191, CD200, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, and CD350.

In certain embodiments, the bispecific agent or antibody disclosed herein binds to two or more markers expressed on human hematopoietic stem cells and selected from the group consisting of CD11a, CD18, CD37, CD47, CD52, CD58, CD62L, CD69, CD74, CD97, CD103, CD132, CD156a, CD179a, CD179b, CD184, CD232, CD244, CD252, CD302, CD305, CD317, and CD361.

In some embodiments, the bispecific antibodies disclosed herein binds to CD34. In some embodiments, the bispecific antibodies disclosed herein binds to CD45. In some embodiments, the bispecific antibodies disclosed herein binds to CD34 and CD45.

In certain embodiments, it may be desirable to use an antibody fragment, rather than an intact antibody. Various techniques are known for the production of antibody fragments. Traditionally, these fragments are derived via proteolytic digestion of intact antibodies (e.g. Morimoto et al., “Single-step Purification of F(ab′)2 Fragments of Mouse Monoclonal Antibodies (immunoglobulins G1) by Hydrophobic Interaction High Performance Liquid Chromatography Using TSKgel Phenyl-5PW,” Journal of Biochemical and Biophysical Methods 24:107-117 (1992) and Brennan et al., “Preparation of Bispecific Antibodies by Chemical Recombination of Monoclonal Immunoglobulin G1 Fragments,” Science 229:81-3 (1985), which are hereby incorporated by reference in their entirety). However, these fragments are now typically produced directly by recombinant host cells as described above. Thus Fab, Fv, and scFv antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870 to Rinderknecht et al., which is hereby incorporated by reference, and can be monospecific or bispecific. Other techniques for the production of antibody fragments will be apparent to the skilled practitioner.

The present invention further encompasses variants and equivalents which are substantially homologous to the chimeric, humanized and human antibodies, or antibody fragments thereof. These can contain, for example, conservative substitution mutations, (e.g., the substitution of one or more amino acids by similar amino acids, which maintain or improve the binding activity of the antibody or antibody fragment).

In a preferred embodiment, cells which express the marker can be used as an immunogen or in a screen for antibody which binds the marker. In one embodiment, the antibody has specificity for the marker, epitope or a portion thereof. In those embodiments where the agent is or comprises an antibody, upon identifying and selecting a marker that is expressed on the surface of the cells of the target tissue (e.g., CD45 or portions or epitopes thereof), an antibody may be raised against such marker using art-recognized techniques and methods.

In certain aspects, the agent is or comprises a ligand. For example, in certain embodiments the agent is or comprises a ligand that interacts or binds to a cell surface receptor.

In certain embodiments, the agent is used to deliver, or to facilitate the delivery of a toxin to the cells of a target tissue and, following the delivery of such toxin to the cells of the target tissue, such toxin is internalized by such cells and thereby exerts a cytotoxic effect on such cells of the target tissue. In certain embodiments, the agent is used to deliver, or to facilitate the delivery of a pore-forming moiety, such as the mutant protective antigen (mut-PA) to the cells of the target tissue. In certain embodiments, upon delivery of an agent coupled to a toxin (e.g., CD45-SAP) to the cells of a target tissue, both the agent and toxin are co-localized to an intracellular compartment of one or more cells of the target tissue, thereby ablating or depleting such cells.

In certain embodiments, the compositions and methods disclosed herein may be administered or otherwise practiced alone or in combination with other available therapies. For example, the methods, conjugates and compositions disclosed herein may be administered to a subject as a primary therapy or as an adjunct therapy.

In certain embodiments, the methods and compositions disclosed herein are practiced or administered in combination with (e.g., co-administered with) one or more mobilizing agents that are capable of inducing the migration of, for example, hematopoietic stem cells and/or progenitor cells from a first compartment (e.g., a target tissue, such as the stem cell niche or the bone marrow compartment) into a second compartment (e.g., the peripheral blood or an organ, such as the spleen), as described in International Publication No. WO2014/134539, the contents of which are incorporated herein by reference in their entirety. In such embodiments, the subject may undergo mobilization therapy, and the agents disclosed herein may be co-administered or subsequently administered to the subject such that the mobilized cells contact the administered composition in the compartment into which such cells were mobilized (e.g., in the peripheral compartment).

In certain aspects, the co-administration of the compositions disclosed herein with one or more mobilizing agents provides a means of increasing or enhancing the activity and/or efficacy of such compositions by increasing the likelihood that the compositions contact, for example, hematopoietic stem cells and/or progenitor cells that have been mobilized into a peripheral compartment. Exemplary, mobilizing agents include, for example one or more of a CXCR2 agonists (e.g., Gro-beta or Gro-betaA4) and a CXCR4 antagonist (e.g., Plerixafor or Mozobil®). In certain aspects, the mobilizing agent comprises, G-CSF alone, or in combination with Plerixafor. In certain aspects, the mobilizing agent comprises at least one heparan sulfate inhibitor. In certain aspects, the mobilizing agent is or comprises filgrastim (GCSF).

In certain embodiments, the cytotoxicity of the methods, compositions and toxins disclosed herein are internalization dependent and thus require the translocation of the toxin into an intracellular compartment of the cells of the target tissue. Such internalization dependent toxicity is distinguishable from previous approaches of targeting using an anti-CD45 radioimmunotoxin (RIT). In particular, by causing such a CD45-RIT to bind specifically to hematopoietic cells, death is not internalization dependent, but rather occurs in nearby cells exposed to irradiation, including undesired irradiation to the spleen and liver. In contrast, the compositions and methods disclosed herein enable CD45 receptor internalization-mediated death using, for example an anti-CD45-SAP immunotoxin. In some embodiments, the methods and compositions disclosed herein do not induce cell death through DNA-damage.

As used herein the terms “internalized” and “internalization” generally mean that the agent and/or toxin are introduced into or otherwise reach the intracellular compartment of one or more cells (e.g., HSCs or progenitor cells) of the target tissue (e.g., bone marrow). For example, an agent and/or toxin may reach the intracellular compartment of a cell via a receptor-mediated process (e.g., an endocytic process) in which the cell will only take in an extracellular agent and/or toxin upon binding to a specific receptor. In certain aspects, the agents and/or toxins disclosed herein are internalized by the endogenous stem cell (e.g., HSCs) or progenitor cell population at a rate of at least about 10%, at least about 15%, at least about 20%, at least about 25%, at least about 30%, at least about 40%, at least about 50%, at least about 60%, at least about 70%, at least about 80%, at least about 90%, at least about 95%, or least about 99%.

In certain aspects, the compositions disclosed herein (e.g., antibody-toxin conjugates) are internalized by a cell expressing a marker (e.g., a CD34 or CD45 cell surface marker) upon binding of such agent (e.g., an antibody) to an epitope of the marker (e.g., CD34 or CD45).

In certain embodiments, the compositions and methods disclosed herein induce cytotoxicity or cell death upon internalization of a toxin or an immunotoxin by a targeted cell (e.g., a hematopoietic stem cell). As used herein, the term “toxin” is used generally to refer to any chemical or biological compound, composition or moiety that can induce a cytotoxic or deleterious effect on a targeted cell. In certain embodiments, the cytotoxic or deleterious effects that are induced by the toxin or immunotoxin occur following its internalization into an intracellular compartment of a cell (e.g., a CD45+ cell). For example, in certain aspects, upon internalization of the agent coupled to the toxin, the toxin is cleaved from the agent (e.g., the toxin and agent are uncoupled) and the toxin inhibits protein synthesis, thereby causing cellular death. Similarly, in certain aspects, upon internalization of the agent coupled to the toxin, the toxin is cleaved from the agent (e.g., the toxin and agent are uncoupled) and the toxin inhibits ribosomal activity, thereby causing cellular death.

Preferably, the toxin must gain cellular entry or otherwise be internalized to exert its cytotoxic or deleterious effect. Accordingly, preferred are toxins that only exert a cytotoxic or deleterious effect following their internalization by one or more cells of the target tissue. Saporin, a catalytic N-glycosidase ribosome-inactivating protein (RIP) that halts protein synthesis, represents an exemplary toxin for use in accordance with the methods and compositions disclosed herein. Unlike other ricin family members, saporin lacks a general cell entry domain and is non-toxic unless coupled to a targeting antibody or ligand that is capable of receptor-mediated internalization. In contrast, when a saporin toxin was coupled to an anti-CD45 antibody, that CD45-SAP conjugate demonstrated 98% depletion of hematopoietic stem cells in bone marrow harvested 8 days post-conditioning. In certain aspects the toxin is coupled to an agent (e.g., a humanized antibody) to facilitate the targeted delivery of such toxin to one or more target cells (e.g., CD45+ cells).

In certain aspects, the toxin is a protein-based toxin, and may include, for example, modified ricin and Ricin A chain derivatives (e.g., Ricin A chain, deglycosylated Ricin A chain), saporin, diphtheria toxin, pseudomonas toxins and variants (e.g. PE38 and others) and small molecule toxins. A toxin can be a protein-based toxin including, for example, biologically-active toxins of bacterial, fungal, plant or animal origin and fragments thereof. In some embodiments, the toxin may be recombinantly-prepared. In certain aspects, a toxin may be a synthetic toxin.

While certain embodiments disclosed herein relate to the use of saporin as the selected toxin, it should be understood that the inventions disclosed herein are not limited to saporin or to protein-based toxins. Rather, several alternative toxins may be used in accordance with the teachings of the present inventions. For example, diphtheria toxin (DT) and pseudomonas exotoxin A (PE) both halt protein synthesis at the elongation step. Ricin family toxins (e.g. saporin) have N-glycosidase activity resulting in the depurination of a critical adenine in the 28S ribosomal RNA (rRNA). All of these toxins inhibit protein synthesis and have the common property of being effective against dividing and non-dividing cells if internalized; this is in contrast to antibody-drug conjugates (ADCs), in which the drugs specifically affect dividing cells by covalently modifying DNA or disrupting microtubule dynamics. As hematopoietic stem cells are normally in a non-proliferating quiescent state, the use of protein toxins capable of inducing cell death regardless of cell-cycle status is preferred for effective hematopoietic stem cell depletion and conditioning. In certain embodiments, the toxin is selected from the group of toxins consisting of saporin, diphtheria toxin, pseudomonas exotoxin A, modified ricin analogs and Ricin A chain derivatives, small molecule toxins and combinations thereof. In certain aspects, the toxin is a modified ricin analogs or Ricin A chain derivatives, for example the ricin A chain. In certain aspects, the toxin (e.g., the ricin A chain) has been modified, for example, to delete a cellular entry domain.

In certain embodiments, the toxin comprises Shiga-like toxin or a subunit thereof, for example, Shiga-like toxin chain A subunit, which is the subunit that is responsible for the toxic action of the Shiga-like toxin protein and is generated by some strains of Escherichia coli. When the protein is inside the cell, the A subunit interacts with the ribosomes to inactivate them, arresting protein synthesis and resulting in apoptosis.

In certain embodiments, the toxin comprises bouganin, which is also a ribosome inactivating protein from the plant Bougainvillea spectabilis. Bouganin is a 29 kDa single-chain type I ribosome-inactivating protein that is able to arrest protein synthesis by the deadenylation of ribosomal RNA resulting in apoptosis.

In certain aspects, the toxin is selected from the group of toxins consisting of abrin toxin, modeccin toxin, gelonin toxin, momordin toxin, trichosanthin toxin, luffin toxin and combinations thereof.

While in certain aspects, the toxin may be a protein-based toxin, it should be understood that the contemplated toxins are not limited to protein-based toxins. Rather, contemplated toxins for use in accordance with any aspects of the present inventions broadly include any compounds or agents (e.g., cytotoxic compounds or agents) that selectively result in the death of one or more cells in the target tissue (e.g., the bone marrow stem cell niche) or that otherwise decrease cell viability. In various embodiments of any aspect of the present inventions, the toxins useful in accordance with the compositions and methods of the present invention comprise one or more DNA-damaging molecules. For example, the selected toxin may comprise one or more anti-tubulin agents (e.g. maytansines) or tubulin inhibitors, DNA crosslinking agents, DNA alkylating agents and cell cycle or mitotic disrupters. In certain aspects, the selected toxin is or comprises a mitotic disruptor or inhibitor, such as maytansine or a functional fragment, derivative or analog thereof.

In certain embodiments, the toxin (e.g., a toxin of fungal origin) inhibits RNA polymerase II and/or III (e.g., an inhibitor of mammalian RNA polymerase II and/or III). In certain aspects such an RNA polymerase II inhibitor toxin is or comprises one or more amatoxins or a functional fragment, derivative or analog thereof. Amatoxins are potent and selective inhibitors of RNA polymerase II, and include all cyclic peptides composed of eight amino acids as isolated from the genus Amanita, most notably Amanita phalloides. Such amatoxins may be isolated from a variety of mushroom species (e.g., Amanita phalloides, Galerina marginata and Lepiota brunneo-incarnata) or in certain aspects may be prepared synthetically. Exemplary toxins suitable for use in accordance with any of the methods or compositions disclosed herein may include or comprise one or more amatoxins selected from the group consisting of α-amanitin, β-amanitin, γ-amanitin, £-amanitin, amanin, amaninamide, amanullin, amanullinic acid and any functional derivatives or analogs thereof. In certain embodiments, the toxin is or comprises α-amanitin, which is an inhibitor of RNA polymerase II and III, or a functional fragment, derivative or analog thereof.

In certain embodiments, the toxin is a small molecule toxin. Such small molecule toxins may be coupled to an agent (e.g., a monoclonal antibody) to form an antibody-drug conjugate (ADC) that may be used, for example, to condition a subject's tissues for engraftment. In certain embodiments, the toxin is derived from bacteria. In some embodiments, the toxin is derived from an insect. In some embodiments, the toxin comprises or is derived from a virus. In some embodiments, the toxin is derived from a plant or a fungus. In some embodiments, the toxin is a naturally-occurring toxin or a fragment thereof. In some embodiments, such a naturally-occurring toxin may be modified relative to its naturally-occurring counterpart, for example, to remove any domains or regions that would facilitate cellular entry or to substitute one or more amino acids.

In certain embodiments, the toxin may be directly coupled or otherwise bound to an agent (e.g., an antibody that specifically or selectively binds CD34 or CD45). For example, the agent is directly coupled to one or more toxins (e.g., as a chimeric fusion protein). As used herein, the terms “couple” and “coupling” broadly refer to any physical, biological or chemical linking or joining of two or more moieties or components together. Such a coupling may be direct or indirect. For example, disclosed herein are agents (e.g., bispecific agents) that may be directly or indirectly coupled to toxins. Similarly, also disclosed are mutant protective antigens (mut-PA) that may be coupled to an agent. Also disclosed is a factor (e.g., lethal factor N-terminus (LFN) and/or edema factor N-terminus (EFN)) that may be coupled to a toxin. In certain embodiments, the factor is or comprises an enzymatic factor.

In certain aspects, the term coupling refers to a functional coupling. For example, contemplated herein are any couplings of two or more moieties that functions to facilitate the co-delivery of such coupled moieties intracellularly. In certain aspects, such a coupling may be direct coupling or an indirect coupling. In certain embodiments, such a coupling may be permanent or temporary. For example, in certain aspects, upon internalization of an agent (e.g., a bispecific agent) coupled to a toxin, the coupling is cleaved, thereby releasing the toxin intracellularly and exerting a cytotoxic effect on the cell.

The agents and the toxin are covalently or non-covalently coupled or linked to each other. Such a coupling may be direct or indirect. For example, a toxin selected from the group of toxins consisting of saporin, diphtheria toxin, pseudomonas exotoxin A, modified ricin analogs and combinations thereof may be directly or indirectly coupled to an antibody that selectively binds CD45 to form an immunotoxin. In some embodiments, the toxins disclosed herein may be indirectly coupled to an antibody. Such antibodies may be biotinylated and coupled to a streptavidin-toxin moiety. Alternatively, in certain embodiments, the toxin may be biotinylated, which may be indirectly coupled to an anti-CD34 or an anti-CD45 antibody that may be bound to or labeled with one or more of streptavidin, avidin, neutravidin and any other variants thereof. In certain aspects, the antibodies disclosed herein are humanized.

In certain aspects, the ratio of agent (e.g., antibody):toxin is about 0.1:1, about 0.25:1, about 0.5:1, about 1:1, about 2:1, about 3:1, about 4:1, about 5:1, about 6:1, about 7:1, about 8:1, about 9:1 or about 10:1. In any of the foregoing embodiments, such ratios are expressed as a ratio of a streptavidin tetramer-toxin chemical conjugate (e.g., a streptavidin tetramer-saporin chemical conjugate). For example, such a streptavidin tetramer may comprise an average of 2.8 toxin (e.g., saporin) molecules and may be expressed as a 1:1 ratio of agent to tetramer-toxin, or alternatively as a 1:2.8 ratio of agent to toxin. In certain embodiments, the ratio of agent (e.g., antibody) to toxin is about 1:2, about 1:2.5, about 1:2.8, about 1:3, about, about 1:3.5, about 1:4, about 1:4.5, about 1:5, about 1:6, about 1:7, about 1:8, about 1:9 or about 1:10. Also contemplated are chimeras, where an antibody and toxin are expressed recombinantly as a single protein. Also contemplated are regions or fragments of antibodies, for example, scFv-toxin conjugate, scFv-toxin chimeras, scFv-toxin multivalent forms that may promote internalization by CD45 receptor cross-linking (e.g., diabodies, tandem di-scFv, tandem tri-scFv, triabodies and/or tetrabodies). Also contemplated are antibody drug conjugates (e.g., CD45-ADCs), which may also be useful for hematological malignancies as an alternative to transplant and, based on the present disclosures concerning the internalizing activity of a cell surface marker (e.g., the CD45 receptor). In certain embodiments, such agents or antibodies are bispecific and bind two cell surface markers.

In certain aspects, the immunotoxins disclosed herein may be prepared by conjugating or coupling a primary antibody to a secondary antibody/toxin conjugate. In such embodiments, the primary antibody recognizes and binds to a marker (e.g., CD45), while the secondary antibody, which is conjugated to a toxin (e.g., saporin), binds to the primary antibody. Thus in such embodiments, the secondary antibody is “piggybacked” onto the primary antibody. Such a secondary antibody may recognize and bind to the heavy chain of the primary antibody and, in certain embodiments, upon binding of the primary antibody to a marker, the immunotoxin construct comprising both the primary and secondary antibodies is internalized by cells expressing such marker. In some embodiments, such immunotoxin constructs may be used to screen for internalization of a primary antibody. In some embodiments, such immunotoxin constructs may be used to assess the toxicity of the immunotoxins disclosed herein and/or to demonstrate the feasibility or viability of, for example, targeting one or more cell surface markers to internalize a toxin. In yet other embodiments, the primary and secondary antibodies may be used in vitro to confirm the desired specificity of the primary antibody for one or more markers (e.g., CD45).

In certain embodiments, the inventions disclosed herein relate to internalizing (antibody fragment) Fab-toxin conjugates. In certain embodiments, the inventions disclosed herein relate to internalizing (single chain fragment) scFv-toxin conjugates. In certain embodiments, the inventions disclosed herein relate to diabody: non-covalent dimer of single-chain Fv (scFv): targeting one or multiple receptors. In certain embodiments, the inventions disclosed herein relate to bivalent (or bispecific) (scFv)₂. In certain embodiments, the inventions disclosed herein relate to tandem scFv. Also contemplated are internalizing aptamer-toxin conjugates and internalizing ligand-toxin conjugates, or any chimeric or non-covalent combination of the above (e.g. scFv-ligand-toxin), as well as all non-covalent formulations (e.g., biotin-streptavidin and including the streptavidin analogs neutravidin and avidin), and chimeric molecules that may be created by recombinant expression of fusion proteins, native chemical ligation, enzyme catalyzed conjugation (e.g. sortase and others) or other conjugation methods (e.g., click chemistry using unnatural amino acids, 1\11-1S-ester agents to modify lysines, maleimide agents to modify cysteine, disulfide bridges). Also contemplated is the incorporation of peptide sequences (e.g., natural, unnatural and cyclic peptides) that facilitate internalization (e.g., HIV-TAT, penetratin, RGD peptide, poly arginine and variants) of the agents and/or toxins disclosed herein.

The methods disclosed herein are not limited to receptor-mediated internalization of a toxin, but rather contemplate any available means of selectively delivering a toxin to an intracellular compartment of the cells of a target tissue. For example, in certain embodiments, disclosed herein are methods of delivering toxins intracellularly using pore-mediated internalization.

Disclosed herein are methods of conditioning a subject for engraftment or methods of selectively depleting or ablating an endogenous stem cell population in a target tissue (e.g., bone marrow tissue) of the subject by administering to the subject an effective amount of a pore-forming chimera comprising a mutant protective antigen (mut-PA) coupled to an agent (e.g., a ligand such as stem cell factor). Protective antigen (PA) is secreted by Bacillus anthracis as water-soluble precursor form PA83 (83 kDa) that undergoes proteolytic activation by furin-type proteases to cleave a 20 kDa fragment off the N-terminus and thereby form the activated PA monomer is able to form pre-pore heptamers. Such a pore-forming chimera forms one or more pores in the cell membrane of the endogenous stem cell population and thereby facilitates the delivery of a subsequently-administered or co-administered toxin to such stem cell population. For example, an effective amount of a second chimera comprising a factor (e.g., an enzymatic factor such as lethal factor N-terminus and/or edema factor N-terminus, or fragments thereof) coupled to a toxin may be administered to the subject, following which the toxin is internalized by the endogenous stem cell population, thereby selectively depleting or ablating the endogenous stem cell population in the target tissue and conditioning the subject for engraftment. In certain embodiments, the factor is lethal factor N-terminus (LFN), or a fragment thereof. In certain embodiments, the factor is edema factor N-terminus (EFN), or a fragment thereof. Both lethal factor (LF) and edema factor (EF) need the binding component protective antigen (PA) for delivery into the cytosol of the cells, where they exhibit enzymatic activities. The 63 kDa C-terminal part of PA forms heptameric channels that inserts in endosomal membranes at low pH, necessary to translocate EF and LF into the cytosol of target cells.

In certain embodiments, a pore-forming moiety, such as the mutant protective antigen (mut-PA), is coupled to an agent that is useful for selectively targeting or directing such pore-forming moiety to the cells of the target tissues (e.g., hematopoietic stem cells or progenitor cells) (Janowiak, B. E., et al., Protein Sci. 18(2): 348-358 (2009); Mourez M. et al., PNAS 100(24): 13803-08 (2003); Ming, Y & R Collier, J. Mol Med. 9(1-2): 46-51 (2003); Rogers M. S., et al., Cancer Res. 15; 67(20):9980-5 (2007)). For example, mutant protective antigens (mut-PA) may be coupled or otherwise fused to agents (e.g., ligands or scFv) to create chimeras that enable the cell-specific forming of cell surface pores. Similarly, in certain embodiments, mutant protective antigens (mut-PA) may be coupled or otherwise fused to a bispecific agent (e.g., a bispecific antibody) to create chimeras that enable the cell-specific forming of cell surface pores. Such cell surface pores may in turn be used or exploited to import or internalize an administered (e.g., co-administered or subsequently-administered) lethal factor N-terminus-toxin chimera (LFN-toxin) and thereby ablate or deplete the cells of the target tissue.

Accordingly, in certain embodiments of the present inventions, the selected toxin may comprise one or more lethal factors coupled (e.g., functionally coupled) to the toxin (e.g., LFN-SAP). Various toxins can be coupled to LFN, including diptheria toxin and/or saporin toxin (e.g., LFN-DTA, LFN-SAP, etc.) In contrast to certain embodiments disclosed herein, the foregoing embodiments advantageously do not require an internalizing marker, receptor or internalizing properties of antibody/ligand, but rather rely on the interaction of PA and LFN to facilitate the delivery of the toxin intracellularly. In some embodiments, the agent is selected from the group consisting of a scfv, a Fab, a discfv, a biscFv, a tri-scfv, a tandem scfv, an aptamer, an antibody and a ligand.

The methods and compositions disclosed herein may be used to condition any number of target tissues of a subject, including, for example bone marrow tissue. As used herein, the term “target tissue” generally refers to any tissues of a subject to which the compositions and methods disclosed herein may be selectively targeted. In certain embodiments, such target tissues comprise an endogenous population of HSCs or progenitor cells (e.g., the stem cell niche of the bone marrow tissue). In certain embodiments, the target tissue is or comprises a subject's bone marrow tissue.

In certain aspects, the compositions and methods of the present inventions are useful for non-myeloablative conditioning in a subject, for example, bone marrow conditioning in advance of hematopoietic stem cell or progenitor cell transplantation. By selectively targeting a marker (e.g., a CD45 cell surface marker) with a toxin (e.g., saporin) that requires cellular entry to exert its cytotoxic effect, the present inventions minimize the incidence and severity of adverse effects. For example, the incidence and severity of adverse effects commonly associated with traditional conditioning regimens, such as mucositis, which may be minimized or in certain instances eliminated. Similarly, the present inventors have demonstrated that conditioning a subject using the methods and compositions (e.g., CD45-SAP immunotoxins) disclosed herein minimizes the incidence of life-threatening thrombocytopenia, neutropenia and red blood cell loss, all of which are commonly associated with traditional conditioning methods, which often require both irradiation and cytotoxic drugs. Accordingly, in certain aspects the compositions and methods disclosed herein are characterized as being non-myeloablative.

The lack of neutropenia observed following conditioning with CD45-SAP and the observed expansion of neutrophils was a surprising result considering neutrophils express CD45. Without wishing to be bound by any particular theory, it may be possible that neutrophils, unlike other blood cells, do not internalize the CD45-SAP or, because of their short life-span (12 hours), that this effect is not visible due to quick turnover of the cell population. It is conceivable that the rapid expansion of neutrophils observed may be a response to CD45+ cell death, as neutrophils are responsible for clearance of apoptotic cells. It is not anticipated that the transient expansion of neutrophils will be an adverse effect, as neutrophils play a prominent role in fighting bacterial infections and their expansion will therefore limit the incidence of bacterial infection, a major cause of traditional conditioning-related mortality.

Although transient lymphopenia in B- and T-cells was observed, it may be that this is necessary (but perhaps not sufficient in itself) for engraftment to occur. While T-cell depletion may be an area of concern for HIV subjects, the transient nature of depletion may be acceptable on a case-by-case assessment of individual patients (especially prior to development of full-blown AIDS). Also, depletion of recipient T-cells may be advantageous as it would enable clearance of CCR5 positive T-cells which serve as viral reservoirs of HIV. The present inventors do not anticipate the transient T-cell depletion to be an issue for the treatment of other hemoglobinopathies, and it is important to note that current conditioning regimens fully ablate T-cell and B-cell populations.

The lack of anemia following CD45-SAP conditioning as evidenced by no decreases in red blood cells, hematocrit or hemoglobin levels, suggests that conditioning in accordance with the methods disclosed herein will be relevant to enabling transplantation in anemic conditions (e.g. sickle cell, Diamond-Blackfan anemia and thalassemias).

The compositions and methods disclosed herein may be used to treat or cure a subject having a disease (e.g., a stem cell disorder) that may benefit from hematopoietic stem cell or progenitor cell transplantation (e.g., sickle cell disease), including, for example autologous, allogeneic, gene-modified and gene-therapy methods. As used herein, the phrase “stem cell disorder” broadly refers to any disease, disorder or condition that may be treated or cured by conditioning a subject's target tissues, and/or by ablating an endogenous stem cell population in a target tissue (e.g., ablating an endogenous HSC or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the present inventions. Similarly, in certain aspects, the compositions and methods disclosed herein may be used for conditioning a subject undergoing treatment for a hematological malignancy. In certain aspects, the methods and compositions disclosed herein may be used to treat, cure or correct diseases selected from the group consisting of the following diseases: sickle cell anemia, thalassemias, Fanconi anemia, Wiskott-Aldrich syndrome, adenosine deaminase SCID (ADA SCID), HIV, metachromatic leukodystrophy, Diamond-Blackfan anemia and Schwachman-Diamond syndrome. In some embodiments, the subject has or is affected by an inherited blood disorder (e.g., sickle cell anemia) or an autoimmune disorder. In some embodiments, the subject has or is affected by a malignancy. For example, a malignancy selected from the group consisting of hematologic cancers (e.g., leukemia, lymphoma, multiple myeloma, or myelodysplastic syndrome) and neuroblastoma. In some embodiments, the subject has or is otherwise affected by a metabolic disorder. For example, in certain aspects the subject may suffer or otherwise be affected by a metabolic disorder selected from the group consisting of glycogen storage diseases, mucopolysccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper IGM syndrome, Chédiak-Higashi disease, hereditary lymphohistiocytosis, osteopetrosis, osteogenesis imperfect, the storage diseases, thalassemia major, sickle cell disease, systemic sclerosis, systemic lupus erythematosus, multiple sclerosis, juvenile rheumatoid arthritis and those diseases or disorders described in “Bone Marrow Transplantation for Non-Malignant Disease,” ASH Education Book, 2000 (1) 319-338, the contents of which are incorporated herein by reference in their entirety.

In certain aspects, the immunotoxin compositions disclosed herein may be used to induce solid organ transplant tolerance. In such embodiments, the immunotoxin compositions and methods disclosed herein may be used to deplete or ablate a population of cells from a target tissue (e.g., to deplete HSCs from the bone marrow stem cell niche). Following such depletion of cells from the target tissues, a population of stem or progenitor cells from the organ donor (e.g., HSCs from the organ donor) may be administered to the transplant recipient and following the engraftment of such stem or progenitor cells, a temporary of stable mixed chimerism achieved, thereby enabling long-term transplant organ tolerance without the need for further immunosuppressive agents. For example, the immunotoxins and methods disclosed herein may be used to induce transplant tolerance in a solid organ transplant recipient (e.g., a kidney transplant, lung transplant, liver transplant and heart transplant). The immunotoxins and methods disclosed herein are well-suited for use in connection the induction of solid organ transplant tolerance, particularly because a low percentage temporary or stable donor engraftment is sufficient to induce long-term tolerance of the transplanted organ.

The methods and compositions disclosed herein are characterized by their enhanced or improved engraftment efficiency. As used herein, the phrases “engraftment efficiency” and “efficiency of engraftment” generally refer to the efficiency with which an administered stem cell population (e.g., HSCs) engrafts in the conditioned target tissue of the subject. In certain embodiments, the efficiency of engraftment is increased by at least about 5%, 7.5%, 10%, 12.5%, 15%, 20%, 25%, 30%, 35%, 40%, 50%, 60%, 70%, 75%, 80%, 90%, 95%, 100% or more. In certain aspects, the determination of engraftment efficiency is assessed relative to the engraftment efficiency of a method in which the engraftment is performed without the conditioning methods disclosed herein.

In some embodiments, the stem cell population (e.g., an exogenous stem cell population) is administered to the target tissues of the subject after the toxin or immunotoxin (e.g., an anti-CD45-SAP immunotoxin) has cleared or dissipated from the subject's target tissues. By allowing the toxin or immunotoxin to clear or to otherwise be reduced to undetectable levels in the subject's target tissues, the ability of any lingering toxin or immunotoxin to exert a cytotoxic effect on the administered stem cell population may be reduced or otherwise eliminated, thereby further increasing the engraftment efficiency of the methods and compositions disclosed herein. Accordingly, in some embodiments, the stem cell population is administered to the subject after the concentration of the immunotoxin in the subject's target tissue has been reduced to an undetectable concentration. The period of time necessary to clear the toxin or immunotoxin from the subject's target tissue may be determined using routine means available to one of skill in the art, for example, by detecting the concentration of the agent, toxin or immunotoxin in the subject's targeted tissue. In addition, the period of time necessary to clear the toxin or immunotoxin from the target tissue be influenced by, or otherwise determined with reference to, among other things, the properties of the agent, toxin or immunotoxin, the administered does of the agent, toxin or immunotoxin, the subject's condition and/or co-morbidities (e.g., renal insufficiency) and the subject's target tissue. For example, in some embodiments, the stem cell population is administered to the target tissue of the subject at least one, two, three, four, five, six, seven, ten, twelve, fourteen, twenty one, thirty six, forty two, fifty six, sixty three, seventy, eighty, ninety, one hundred, one hundred and twenty days, six months, nine months, twelve months, or more, after the immunotoxin has cleared or dissipated from the target tissues of the subject.

As used herein, the term “subject” refers to an animal, for example, a mammal or a human to whom the treatments disclosed herein may be provided. For treatment of those disease states which are specific for a specific animal such as a human subject, the term subject refers to that specific animal. In certain embodiments, the subject is a human (e.g., an adolescent, adult or an elderly human).

The compositions of the present invention may be prepared and pharmaceutically acceptable carriers and excipients selected, as described in detail in, for example, L. William, Remington: The Science and Practice of Pharmacy. 22nd ed. Pharmaceutical Press (2012), the entire contents of which are incorporated herein by reference. In certain aspects, the compositions disclosed herein (e.g., a CD45-SAP conjugate) are formulated for parenteral administration to a subject.

As used herein, the term “effective amount” means an amount sufficient to achieve a meaningful benefit to the subject (e.g., condition the subject's target tissue for transplant). For example, an effective amount of the agents that are the subject of the present inventions may be generally determined based on the activity of such agents and the amount of such agents that are necessary to ablate or deplete the stem cell niche. An effective amount of the compositions (e.g., antibody-toxin conjugates) necessary to condition the subject or to ablate the subject's hematopoietic stem cells or progenitor cells can be readily determined depending on the subject's disease and other related characteristics. Such characteristics include the condition, general health, age, subjective symptoms, objective appearance, sex and body weight of the subject.

In some embodiments, an effective amount of the immunotoxin compositions disclosed herein achieves maximal stem cell depletion (e.g., about 90%, 91%, 92%, 93%, 94%, 95%, 96%, 97%, 97.5%, 98%, 99%, 99.5% or more depletion of hematopoietic or progenitor stem cells from the target tissues of the subject). In some embodiments, an effective amount of the compositions disclosed herein is determined on the basis of a subject's weight. For example, in certain aspects, such an effective amount of the compositions disclosed herein is or comprises one or more doses of ranging between about 10-0.01 mg/kg. In certain aspects, an effective amount of the compositions disclosed herein (e.g., a CD45-toxin conjugate) is or comprises one or more doses of 4.0 mg/kg. In some aspects, an effective amount of the compositions disclosed herein is or comprises one or more doses of 3.0 mg/kg. In certain aspects, an effective amount of the compositions disclosed herein is or comprises one or more doses of 2.0 mg/kg. In some aspects, an effective amount of the compositions disclosed herein is or comprises one or more doses of 2.5 mg/kg. In certain aspects, an effective amount of the compositions disclosed herein is or comprises one or more doses of 2.0 mg/kg. In certain embodiments, an effective amount of the compositions disclosed herein (e.g., a CD45-toxin conjugate) is or comprises one or more doses of 1.5 mg/kg. In certain aspects, an effective amount of the compositions disclosed herein (e.g., a CD45-SAP conjugate) is or comprises one or more doses of 1.0 mg/kg.

Also disclosed herein are methods and assays for identifying candidate agents that may be useful for selectively depleting or ablating an endogenous stem cell population in accordance with the methods disclosed herein. In certain embodiments, such methods comprise a step of contacting a sample (e.g., a sample obtained from a subject) comprising the stem cell population with a test agent coupled to a toxin. Following such a contacting step, a determination is made as to whether one or more cells of the stem cell population are depleted or ablated from the sample, wherein the depletion or ablation of one or more cells of the HSC or progenitor cell population following the contacting step identifies the test agent as a candidate agent which may be useful for selectively depleting or ablating an endogenous stem cell population. In some embodiments, the cell is contacted with the test agent for at least about 2-24 hours or more. As used herein, the terms “contact” and “contacting” refer to bringing two or more moieties (e.g., a cell and an agent) together, or within close proximity of one another such that the moieties may react. For example, in one embodiment the assays of the present invention comprise a step of contacting a stem cell population with a test agent.

It is to be understood that the invention is not limited in its application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain agents, compounds, compositions and methods of the present invention have been described with specificity in accordance with certain embodiments, the following examples serve only to illustrate the methods and compositions of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

### EXAMPLES

### Example 1

The present inventors conducted a KG1a hematopoietic progenitor cell killing assay. Immunotoxins were created using saporin and the listed commercially available anti-human monoclonal antibodies (mAb; purchased from BD Biosciences) targeting various cell surface receptors and were tested for their ability to kill the KG1a hematopoietic progenitor cells over 72 hours. Cell death was assessed by the MTS assay, which measured metabolic activity. As a 100% death control, cells were incubated with 10 μM staurosporine.

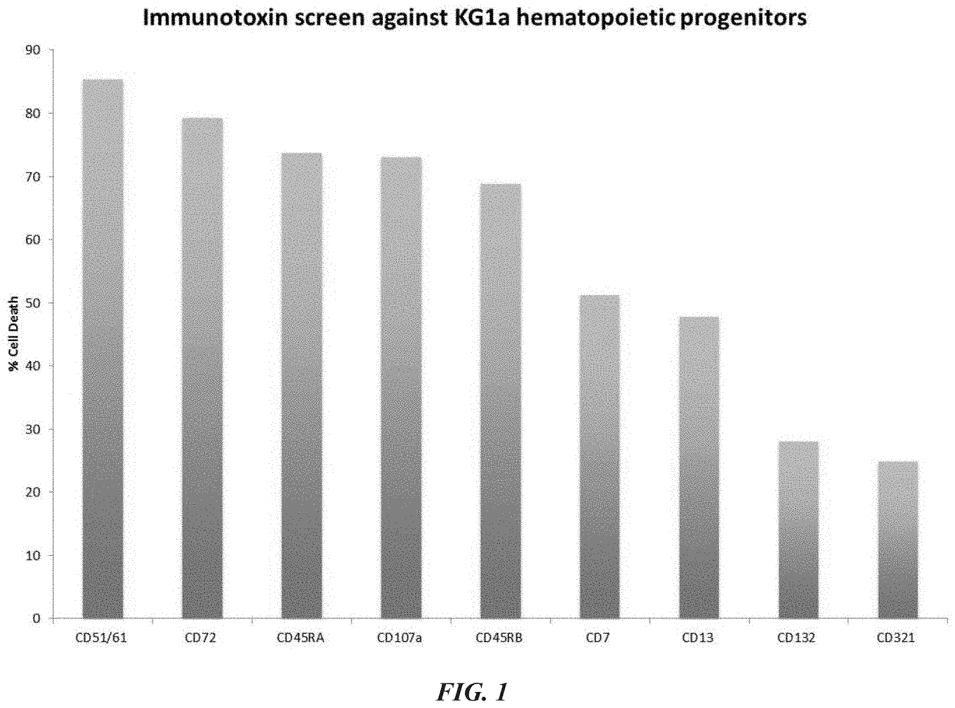

Immunotoxins that killed greater than 20% of the KG1a hematopoietic progenitor cells are shown below in Table 1 below and depicted in FIG. 1.

TABLE 1
KG1a Hematopoietic Progenitor Cell Killing Assay

| | | | Immunotoxin | |
|---|---|---|---|---|
| | | | concentration | |
| Target | Clone | | (nM) | % Cell death |
| CD51/61 | 23C6 | Ms IgG 1, κ | 10 | 85.32423208 |
| CD72 | J4- 117 | Ms IgG 2b, κ | 3 | 79.29824561 |
| CD45RA | HI100 | Ms IgG 2b, κ | 3 | 73.68421053 |
| CD107a | H4A3 | Ms IgG 1, κ | 3 | 73.03754266 |
| CD45RB | MT4 | Ms IgG 1, κ | 3 | 68.77192982 |
| CD7 | M-T701 | Ms IgG 1, κ | 3 | 51.22807018 |
| CD13 | WM15 | Ms IgG 1, κ | 3 | 47.71929825 |
| CD132 | T UGh4 | Rt IgG 2b, κ | 3 | 27.98381659 |
| CD321 | M.AB.F11 | Ms IgG 1, κ | 3 | 24.91349481 |

This data evidences that the immunotoxins of the present invention demonstrate KG1a hematopoietic progenitor cell killing activity.

### Example 2

The present inventors also conducted a primary human bone marrow CD34+ cell killing assay. Immunotoxins were created using saporin and the listed commercially available anti-human monoclonal antibodies (mAb; purchased from BD Biosciences) targeting various cell surface receptors and were tested for their ability to kill the primary human bone marrow CD34+ cells over 120 hours. Cell death was assessed by the MTS assay, which measured metabolic activity. As a 100% death control, cells were incubated with 10 μM staurosporine.

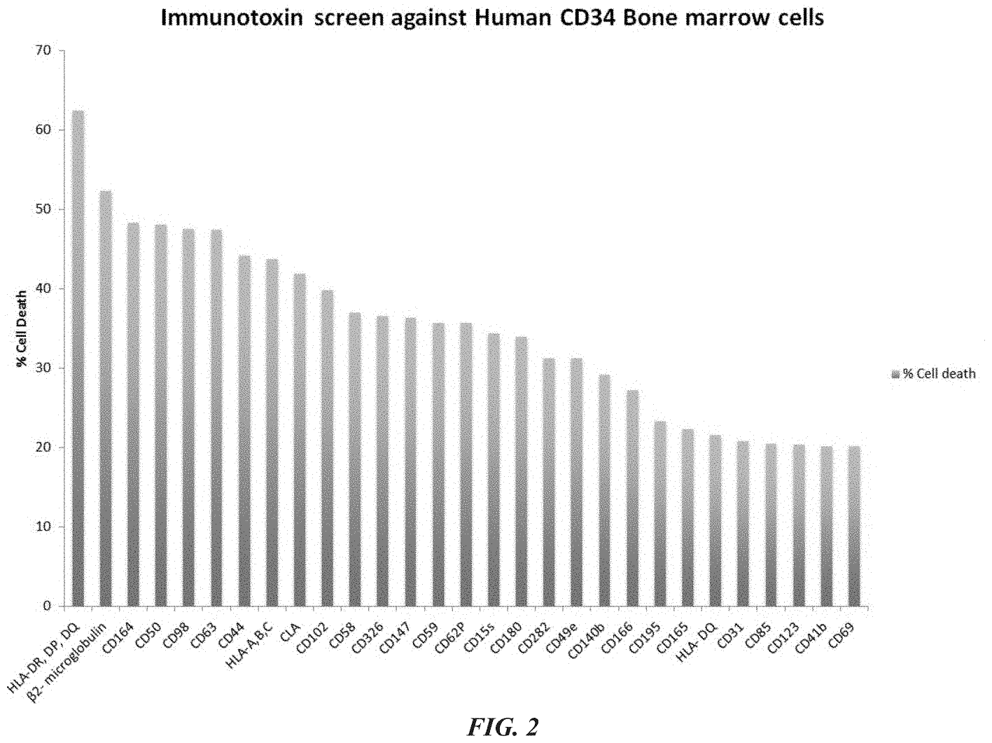

Immunotoxins that killed greater than 20% of the primary human bone marrow CD34+ cells are shown below in Table 2 below and depicted in FIG. 2.

TABLE 2
Primary Human Bone Marrow CD34+ Cell Killing Assay

| | | | Immunotoxin | |
|---|---|---|---|---|
| | | | concentration | |
| Target | Clone | | (nM) | % Cell death |
| HLA-DR, | TU39 | Ms IgG 2a, κ | 10 | 62.3655914 |
| DP, DQ | | | | |
| β2-micro- | TU99 | Ms IgM, κ | 3 | 52.31788079 |
| globulin | | | | |
| CD164 | N6B6 | Ms IgG 2a, κ | 3 | 48.25174825 |
| CD50 | TU41 | Ms IgG 2b, κ | 3 | 48.05194805 |
| CD98 | UM7F8 | Ms IgG 1, κ | 3 | 47.55244755 |
| CD63 | H5C6 | Ms IgG 1, κ | 3 | 47.4025974 |
| CD44 | G44- 26 | Ms IgG 2b, κ | 3 | 44.15584416 |
| HLA-A, | G46- 2.6 | Ms IgG 1, κ | 3 | 43.70860927 |
| B, C | | | | |
| CLA | HECA- 452 | Rt IgM, κ | 10 | 41.81818182 |
| CD102 | CBR- | Ms IgG 2a, κ | 3 | 39.86013986 |
| | 1C2/2.1 | | | |
| CD58 | 1C3 | Ms IgG 2a, κ | 3 | 37.01298701 |
| CD326 | EBA-1 | Ms IgG 1, λ | 10 | 36.55913978 |
| CD147 | HIM6 | Ms IgG 1, κ | 3 | 36.36363636 |
| CD59 | p282 (H19) | Ms IgG 2a, κ | 3 | 35.71428571 |
| CD62P | AK-4 | Ms IgG 1, κ | 3 | 35.71428571 |
| CD15s | CSLEX1 | Ms IgM, κ | 3 | 34.41558442 |
| CD180 | G28- 8 | Ms IgG 1, κ | 10 | 33.98058252 |
| CD282 | 11G7 | Ms IgG 1, κ | 10 | 31.1827957 |
| CD49e | VC5 | Ms IgG 1, κ | 3 | 31.16883117 |
| CD140b | 28D4 | Ms IgG 2a, κ | 10 | 29.12621359 |
| CD166 | 3A6 | Ms IgG 1, κ | 10 | 27.18446602 |
| CD195 | 2D7/CCR5 | Ms IgG 2a, κ | 10 | 23.30097087 |
| CD165 | SN2 | Ms IgG 1, κ | 10 | 22.33009709 |
| HLA- DQ | T U169 | Ms IgG 2a, κ | 10 | 21.50537634 |
| CD31 | WM59 | Ms IgG 1, κ | 3 | 20.77922078 |
| CD85 | GHI/75 | Ms IgG 2b, κ | 3 | 20.44444444 |
| CD123 | 9F5 | Ms IgG 1, κ | 10 | 20.38834951 |
| CD41b | HIP2 | Ms IgG 3, κ | 3 | 20.12987013 |
| CD69 | FN50 | Ms IgG 1, κ | 3 | 20.12987013 |
| CD162 | KPL- 1 | Ms IgG 1, κ | 10 | 66.99029126 |
| CD43 | 1G10 | Ms IgG 1, κ | 10 | 96.55172414 |
| CD71 | M-A712 | Ms IgG 2a, κ | 3 | 95.45454545 |
| CD47 | B6H12 | Ms IgG 1, κ | 3 | 66.88311688 |
| CD97 | VIM3b | Ms IgG 1, κ | 10 | 66.01941748 |
| CD205 | MG38 | Ms IgG 2b | 3 | 65.03496503 |
| HLA- DR | G46-6 | Ms IgG 2a, κ | 3 | 56.95364238 |
| | (L243) | | | |
| CD34 | 581 | Ms IgG 1, κ | 10 | 47.12643678 |
| CD49d | 9F10 | Ms IgG 1, κ | 10 | 47.12643678 |
| CD184 | 12G5 | Ms IgG 2a, κ | 3 | 42.65734266 |
| CD84 | 2G7 | Ms IgG 1, κ | 3 | 33.76623377 |
| CD48 | T U145 | Ms IgM, κ | 3 | 27.92207792 |
| CD11a | G43- 25B | Ms IgG 2a, κ | 3 | 25.32467532 |
| CD62L | Dreg 56 | Ms IgG 1, κ | 3 | 20.77922078 |

### Discussion

Work described herein demonstrates the ability of single-entity immunotoxin agents targeting various cell surface receptors to kill KG1a hematopoietic progenitors and primary human bone marrow CD34+ cells.

The use of protein immunotoxins offers significant advantages as compared to whole body irradiation, DNA-alkylating agents or radioimmunotherapy (RIT). In addition to the specificity that is achieved by antibody targeting, the requirement for receptor-mediated internalization of protein toxin significantly reduces risks of off-target and bystander toxicity (e.g., to niche cells). Protein-based immunotoxins may be preferred for non-malignant conditions where stable mixed chimerism is sufficient to cure the underlying disease (e.g. hemoglobinopathies and SCID conditions). Additionally, the enhanced stability and cost-effective production of protein-based immunotoxins likely facilitates widespread use, especially in countries in which hemoglobinopathies are more prevalent. In addition, as protein-based immunotoxins compared to RIT do not induce DNA-damage, they may be better suited to condition pre-malignant Fanconi Anemia patients, who are genetically predisposed to be hyper-sensitive to DNA damaging agents and conventional conditioning.

Furthermore, the methods and compositions disclosed herein selectively target cells expressing HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11 and/or CD62L, since internalization is a prerequisite for cell death. In contrast, while radioimmunotoxins will bind specifically to cells having the appropriate surface protein, death is not internalization-dependent and will occur in nearby cells exposed to irradiation (including undesired irradiation to the spleen and liver). Importantly, the radiation-exposed cells, which include cells comprising the niche, are essential for engraftment to proceed (Wang, Y., et al., Free Radic Biol Med (2010), 48, 348-356; Wang, Y., et al., Blood (2006), 107, 358-366; and Madhusudhan, T., et al., Stem Cells Dev (2004), 13, 173-182). Therefore, while radioimmunotoxins are suited for BMT in patients with malignancy (e.g., where myeloablative conditioning is necessary to enable 100% donor chimerism), the immunotoxins disclosed herein are suitable for treatment of subjects where partial chimerism is sufficient to correct non-malignant disease and minimize the risks during the conditioning procedure. The reduced risk and, the utility of immunotoxins disclosed herein as a single-entity shelf-stable agent, will likely enable more wide-spread use of bone marrow transplant (both allogeneic and gene therapy autologous) even to hospitals that currently lack the infrastructure (e.g. irradiator) or palliative care facilities to perform traditional BMT.

While the clinical utility of immunotoxins in anti-cancer therapy has largely been limited by issues of immunogenicity and cumulative dose-limiting toxicity, these factors are not applicable to pre-HSC transplant conditioning where non-recurrent use is likely. Furthermore, the wealth of safety data available from previous immunotoxin clinical trials targeting hematological malignancies may in fact facilitate rapid clinical translation. The results presented herein strongly suggest that protein-based immunotoxins as disclosed herein, may be useful in stem cell transplantation to enable the treatment of diseases that are currently limited by toxicities of existing conditioning regimens.

What is claimed is:

1. A method of conditioning a subject for engraftment, the method comprising selectively depleting or ablating an endogenous hematopoietic stem cell (HSC) or progenitor cell population in a target tissue of the subject by administering to the subject an effective amount of an agent coupled to a toxin; wherein the toxin is internalized by the endogenous stem cell population, thereby depleting or ablating the endogenous hematopoietic stem cell or progenitor cell population in the target tissue and conditioning the subject for engraftment; wherein the hematopoietic stem cell or progenitor cell population expresses one or more markers selected from the group of markers consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a, CD62L, CD51/61, CD72, CD45RA, CD107a, CD45RB, CD7, CD13, CD132 and CD321, wherein the agent selectively binds to the one or more markers or a fragment or epitope thereof. and wherein the agent is selected from the group consisting of an antibody and a ligand.

2. A method of engrafting stem cells in a subject, the method comprising: (a) administering to the subject an effective amount of an agent coupled to a toxin, wherein the toxin is internalized by an endogenous hematopoietic stem cell (HSC) or progenitor cell population, thereby selectively depleting or ablating the endogenous hematopoietic stem cell or progenitor cell population in a target tissue of the subject, wherein the hematopoietic stem cell or progenitor cell population expresses one or more markers selected from the group of markers consisting of HLA-DR, HLA-DP, HLA-DQ, 32-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a, CD62L, CD51/61, CD72, CD45RA, CD107a, CD45RB, CD7, CD13, CD132 and CD321, and wherein the agent selectively binds to the one or more markers or a fragment or epitope thereof; and (b) administering a stem cell population to the target tissue of the subject, wherein the administered stem cell population engrafts in the target tissue of the subject.

3. A method of treating a stem cell disorder in a subject, the method comprising: (a) administering to the subject an effective amount of an agent coupled to a toxin, wherein the toxin is internalized by an endogenous hematopoietic stem cell (HSC) or progenitor cell population in a target tissue of the subject, thereby depleting or ablating the endogenous hematopoietic stem cell or progenitor cell population in the target tissue of the subject, wherein the hematopoietic stem cell or progenitor cell population expresses one or more markers selected from the group of markers consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a, CD62L, CD51/61, CD72, CD45RA, CD107a, CD45RB, CD7, CD13, CD132 and CD321, and wherein the agent selectively binds to the one or more markers or a fragment or epitope thereof; and (b) administering a stem cell population to the target tissue of the subject, wherein the administered stem cell population engrafts in the target tissue of the subject.

4. A method of selectively depleting or ablating an endogenous hematopoietic stem cell (HSC) or progenitor cell population in a target tissue of a subject, the method comprising administering to the subject an effective amount of a composition comprising an agent and a toxin; wherein the endogenous HSC or progenitor cell population expresses a marker, and wherein the agent selectively binds to the marker and is internalized by the endogenous HSC or progenitor cell population, thereby depleting or ablating the endogenous HSC or progenitor cell population in the target tissue, wherein the marker is selected from the group of markers consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a, CD62L, CD51/61, CD72, CD45RA, CD107a, CD45RB, CD7, CD13, CD132 and CD321.

5. The method of claims 1-4, wherein the agent is an antibody.

6. The method of claims 1-4, wherein the agent is a ligand.

7. The method of claims 1-6, wherein the toxin is internalized by receptor-mediated internalization.

8. The method of claims 1 and 4, further comprising a step of administering a stem cell population to the target tissue of the subject after the endogenous hematopoietic stem cell or progenitor cell population is depleted or ablated, wherein the administered stem cell population engrafts in the target tissue of the subject.

9. The method of claims 2, 3 and 8, wherein the method increases efficiency of the engraftment of the administered stem cell population in the target tissue, as compared to a method performed using only the step of administering the stem cell population to the target tissue of the subject.

10. The method of claim 9, wherein the efficiency of engraftment is increased by at least about 100%.

11. The method of claims 2, 3 and 8, wherein the stem cell population comprises an exogenous stem cell population.

12. The method of claims 2, 3 and 8, wherein the stem cell population comprises the subject's endogenous stem cells.

13. The method of claim 12, wherein the endogenous stem cells are genetically modified.

14. The method of claims 1-13, wherein the hematopoietic stem cell or progenitor cell population expresses one or more markers selected from the group of markers consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L.

15. The method of claims 1-13, wherein the hematopoietic stem cell or progenitor cell population expresses one or more markers selected from the group of markers consisting of CD51/61, CD72, CD45RA, CD107a, CD45RB, CD7, CD13, CD132 and CD321.

16. The method of claims 1-15, wherein the toxin inhibits protein synthesis and is selected from the group of toxins consisting of Shiga-like toxin chain A, bouganin and combinations thereof.

17. The method of claim 16, wherein the toxin comprises a Shiga-like toxin.

18. The method of claim 17, wherein the Shiga-like toxin comprises Shiga-like toxin chain A.

19. The method of claim 16, wherein the toxin comprises bouganin.

20. The method of claims 1-19, wherein the toxin is internalized at a rate of at least about 10%.

21. The method of claims 1-19, wherein the toxin is internalized by the endogenous stem cell population at a rate of at least about 50%.

22. The method of claims 1-19, wherein the toxin is internalized by the endogenous stem cell population at a rate of at least about 90%.

23. The method of claims 2-3 and 8, wherein the stem cell population is administered to the target tissue of the subject after the toxin has dissipated from the target tissue.

24. The method of claims 1-23, wherein the toxin is selected from the group of toxins consisting of saporin, diphtheria toxin, pseudomonas exotoxin A, Ricin A chain derivatives, Shiga-like toxin chain A, bouganin a small molecule toxin and combinations thereof.

25. The method of claims 1-23, wherein the toxin comprises saporin.

26. The method of claims 1-23, wherein the toxin inactivates ribosomes.

27. The method of claims 1-25, wherein the toxin inhibits protein synthesis.

28. The method of claims 1-27, wherein the toxin is not a radioimmunotoxin.

29. The method of claims 1-28, wherein the agent is directly coupled to the toxin.

30. The method of claims 1-28, wherein the agent is indirectly coupled to the toxin.

31. The method of claim 30, wherein the agent is biotinylated.

32. The method of claim 30, wherein the agent is coupled to a streptavidin-toxin chimera.

33. The method of claims 1-32, wherein the target tissue comprises bone marrow tissue.

34. The method of claims 1-33, wherein the method does not deplete or ablate the subject's endogenous neutrophils.

35. The method of claims 1-34, wherein the method causes an increase in the subject's mature endogenous neutrophils.

36. The method of claims 1-35, wherein the method does not deplete or ablate the subject's endogenous platelets.

37. The method of claims 1-36, wherein the method does not induce anemia in the subject.

38. The method of claims 1-37, wherein the method causes an increase in granulocyte colony stimulating factor (GCSF).

39. The method of claims 1-38, wherein the method causes an increase in macrophage colony stimulating factor (MCSF).

40. The method of claims 1-39, wherein the method causes an increase in the subject's endogenous myeloid cells.

41. The method of claims 1-40, wherein the method does not deplete or ablate the subject's endogenous lymphoid cells.

42. The method of claims 1-41, wherein the method preserves innate immunity of the subject.

43. The method of claim 1-42, wherein the method preserves adaptive immunity of the subject.

44. The method of claims 1-43, wherein the method preserves thymic integrity of the subject.

45. The method of claims 1-44, wherein the method preserves vascular integrity of the subject.

46. The method of claims 2, 3 and 8, wherein the method achieves at least about 90% engraftment of the exogenous stem cell population.

47. The method of claims 2, 3 and 8, wherein the method achieves at least about 20% donor chimerism in the target tissue four months post-administration of the exogenous stem cell population to the subject.

48. The method of claims 1-47, wherein the subject has a non-malignant hemoglobinopathy.

49. The method of claim 48, wherein the hemoglobinopathy is selected from the group consisting of sickle cell anemia, thalassemia, Fanconi anemia, and Wiskott-Aldrich syndrome.

50. The method of claims 1-3, wherein the subject has an immunodeficiency.

51. The method of claim 50, wherein the immunodeficiency is a congenital immunodeficiency.

52. The method of claim 50, wherein the immunodeficiency is an acquired immunodeficiency.

53. The method of claim 52, wherein the acquired immunodeficiency is selected from the group consisting of HIV and AIDS.

54. The method of claim 3, wherein the stem cell disorder is selected from the group of disorders consisting of a non-malignant hemoglobinopathy, an immunodeficiency and cancer.

55. The method of claims 1-47, wherein the subject has a malignant, pre-malignant or non-malignant disorder.

56. The method of claims 1-47, wherein the subject has or is affected by a malignancy selected from the group consisting of leukemia, lymphoma, multiple myeloma, myelodysplastic syndrome and neuroblastoma.

57. The method of claims 1-47, wherein the subject has a disorder selected from the group consisting of a glycogen storage disease, mucopolysccharidoses, Gaucher's Disease, Hurlers Disease, sphingolipidoses, metachromatic leukodystrophy, severe combined immunodeficiency, Wiscott-Aldrich syndrome, hyper IGM syndrome, Chédiak-Higashi disease, hereditary lymphohistiocytosis, osteopetrosis, osteogenesis imperfect, a storage disease, thalassemia major, sickle cell disease, systemic sclerosis, systemic lupus erythematosus, multiple sclerosis, and juvenile rheumatoid arthritis.

58. The method of claims 1-57, wherein the agent is an antibody, and wherein the antibody is selected from the group consisting of clone KPL-1, clone 1G10, clone M-A712, clone B6H12, clone VIM3b, clone MG38, clone G46-6 (L243), clone 581, clone 9F10, clone 12G5, clone 2G7, clone T U145, clone G43-25B and clone Dreg 56.

59. The method of claim 58, wherein the antibody comprises clone 1G10.

60. The method of claim 58, wherein the antibody comprises clone Dreg 56.

61. The method of claims 1-57, wherein the agent comprises an antibody, and wherein the antibody is humanized.

62. The method of claims 1-61, wherein the subject is a mammal.

63. The method of claims 1-62, wherein the subject is a human.

64. The method of claims 1-63, wherein the subject is immunocompetent.

65. The method of claims 1-4, wherein the agent is an antibody selected from the group consisting of clone 23C6, clone J4-117, clone HI100, clone H4A3, clone MT4, clone M-T701, clone WM15, clone TUGh4 and clone M.AB.F11.

66. The method of claims 1-4, wherein the agent is an antibody selected from the group consisting of clone TU39, clone TU99, clone N6B6, clone TU41, clone UM7F8, clone H5C6, clone G44-26, clone G46-2.6, clone HECA-452, clone CBR-1C2/2.1, clone 1C3, clone EBA-1, clone HIM6, clone p282 (H19), clone AK-4, clone CSLEX1, clone G28-8, clone 11G7, clone VC5, clone 28D4, clone 3A6, clone 2D7/CCR5, clone SN2, clone TU169, clone WM59, clone GHI/75, clone 9F5, clone HIP2, clone FN50, clone KPL-1, clone 1G10, clone M-A712, clone B6H12, clone VIM3b, clone MG38, clone G46-6 (L243), clone 581, clone 9F10, clone 12G5, clone 2G7, clone TU145, clone G43-25B and clone Dreg 56.

67. The method of claims 1-4, wherein the agent is an antibody, and wherein the antibody is clone KPL-1.

68. The method of claims 1-4, wherein the agent is an antibody, and wherein the antibody is clone 1G10.

69. The method of claims 1-4, wherein the agent is an antibody, and wherein the antibody is clone M-A712.

70. The method of claims 1-4, wherein the agent is an antibody, and wherein the antibody is clone B6H12

71. The method of claims 1-4, wherein the agent is an antibody, and wherein the antibody is clone VIM3b

72. The method of claims 1-71, wherein the method does not induce cell death through DNA-damage.

73. A method of identifying a candidate agent for selectively depleting or ablating an endogenous stem cell population, the method comprising the steps of: (a) contacting a sample comprising the stem cell population with a test agent coupled to a toxin; and (b) detecting whether one or more cells of the stem cell population are depleted or ablated from the sample; wherein the depletion or ablation of one or more cells of the stem cell population following the contacting step identifies the test agent as a candidate agent, wherein the stem cells comprise hematopoietic stem cells or progenitor cells that express one or more markers selected from the group of markers consisting of CD162, CD43, CD71, CD47, CD97, CD205, HLA-DR, CD34, CD49d, CD184, CD84, CD48, CD11a and CD62L.

74. The method of claim 73, wherein the test agent is an antibody.

75. The method of claim 73, wherein the test agent is a ligand.

76. The method of claim 73, wherein the toxin is internalized by the one or more cells of the HSC or progenitor cell population.

77. The method of claim 77, wherein the internalization comprises receptor-mediated internalization.

78. The method of claims 73-77, wherein the toxin is selected from the group of toxins consisting of saporin, diphtheria toxin, pseudomonas exotoxin A, Ricin A chain derivatives, Shiga-like toxin chain A, bouganin a small molecule toxin and combinations thereof.

79. The method of claims 73-78, wherein the cell is contacted with the test agent for at least about 2-24 hours.

80. The method of claim 73-79, wherein the cell is a human cell.

81. A method of conditioning a subject for engraftment, the method comprising selectively depleting or ablating an endogenous hematopoietic stem cell or progenitor cell population in a target tissue of the subject by: (a) administering to the subject an effective amount of a pore-forming chimera comprising a mutant protective antigen (mut-PA) coupled to an agent, and thereby forming one or more pores in the cell membrane of the endogenous hematopoietic stem cell or progenitor cell population; and (b) administering to the subject an effective amount of a second chimera, wherein the second chimera comprises a lethal factor N-terminus (LFN) coupled to a toxin, and wherein the toxin is internalized by the endogenous hematopoietic stem cell or progenitor cell population, thereby selectively depleting or ablating the endogenous hematopoietic stem cell or progenitor cell population in the target tissue and conditioning the subject for engraftment; wherein the hematopoietic stem cells or progenitor cells comprise or express one or more markers selected from the group of markers consisting of: HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a, CD62L, CD51/61, CD72, CD45RA, CD107a, CD45RB, CD7, CD13, CD132 and CD321, and wherein the agent selectively binds to the marker or a fragment or epitope thereof.

82. A method of engrafting stem cells in a subject, the method comprising: (a) administering to the subject an effective amount of a pore-forming chimera comprising a mutant protective antigen (mut-PA) coupled to an agent, and thereby forming one or more pores in the cell membrane of an endogenous hematopoietic stem cell or progenitor cell population; (b) administering to the subject an effective amount of a second chimera, wherein the second chimera comprises a factor coupled to a toxin, wherein the factor is selected from the group consisting of lethal factor N-terminus (LFN) and edema factor N-terminus (EFN), and wherein the toxin is internalized by the endogenous hematopoietic stem cell or progenitor cell population, thereby depleting or ablating the endogenous hematopoietic stem cell or progenitor cell population in the target tissue; and (c) administering a stem cell population to the target tissue of the subject, wherein the administered stem cell population engrafts in the target tissue of the subject; wherein the hematopoietic stem cells or progenitor cells comprise or express one or more markers selected from the group of markers consisting of: HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a, CD62L, CD51/61, CD72, CD45RA, CD107a, CD45RB, CD7, CD13, CD132 and CD321, and wherein the agent selectively binds to the marker or a fragment or epitope thereof.

83. A method of treating a stem cell disorder in a subject, the method comprising: (a) administering to the subject an effective amount of a pore-forming chimera comprising a mutant protective antigen (mut-PA) coupled to an agent, and thereby forming one or more pores in the cell membrane of an endogenous hematopoietic stem cell or progenitor cell population; (b) administering to the subject an effective amount of a second chimera, wherein the second chimera comprises a factor coupled to a toxin, wherein the factor is selected from the group consisting of lethal factor N-terminus (LFN) and edema factor N-terminus (EFN), and wherein the toxin is internalized by the endogenous hematopoietic stem cell or progenitor cell population, thereby selectively depleting or ablating the endogenous hematopoietic stem cell or progenitor cell population in the target tissue; and (c) administering a stem cell population to the target tissue of the subject, wherein the administered stem cell population engrafts in the target tissue of the subject; wherein the hematopoietic stem cells or progenitor cells comprise or express one or more markers selected from the group of markers consisting of: HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a, CD62L, CD51/61, CD72, CD45RA, CD107a, CD45RB, CD7, CD13, CD132 and CD321, and wherein the agent selectively binds to the marker or a fragment or epitope thereof.

84. The methods of claims 81-83, wherein the toxin is internalized by a pore-mediated internalization.

85. The methods of claims 81-84, wherein the method does not induce cell death through DNA-damage.

86. The method of claims 81-85, wherein the agent is a single-chain variable fragment (scFv).

87. The method of claims 81-86, wherein the agent is a ligand.

88. The method of claim 89, wherein the ligand is selected from the group of ligands consisting of CXCL12: Stromal derived factor 1 (SDF1), Angiopoietin 1 to 4 (Ang1, Ang2, Ang3, Ang4), TPO (thrombopoietin), Erythropoietin, FLT3L, VLA4, VLA6, IL-1, IL-3, IL-6, IL-18, G-CSF, Oncostatin M and LIF.

89. The method of claims 81-83, wherein the agent is selected from the group consisting of a scfv, a Fab, a discfv, a biscFv, a tri-scfv, a tandem scfv, an aptamer, an antibody and a ligand.

90. The method of claim 89, wherein the agent selectively binds to the marker.

91. The method of claim 81-90, wherein the subject is a mammal.

92. The method of claim 81-90, wherein the subject is a human.

93. The method of claims 81-90, wherein the subject has a non-malignant hemoglobinopathy.

94. The method of claim 93, wherein the hemoglobinopathy is selected from the group consisting of sickle cell anemia, thalassemia, Fanconi anemia, and Wiskott-Aldrich syndrome.

95. The method of claims 81-92, wherein the subject has an immunodeficiency.

96. The method of claim 95, wherein the immunodeficiency is a congenital immunodeficiency.

97. The method of claim 95, wherein the immunodeficiency is an acquired immunodeficiency.

98. The method of claim 97, wherein the acquired immunodeficiency is selected from the group consisting of HIV and AIDS.

99. The method of claim 83, wherein the stem cell disorder is selected from the group of disorders consisting of a non-malignant hemoglobinopathy, an immunodeficiency and cancer.

100. The method of claims 81-99, wherein the toxin is selected from the group of toxins consisting of saporin, diphtheria toxin, pseudomonas exotoxin A, Ricin A chain derivatives, Shiga-like toxin chain A, bouganin, a small molecule toxin and combinations thereof.

101. The method of claims 81-99, wherein the toxin comprises saporin.

102. The method of claims 81-99, wherein the toxin inactivates ribosomes.

103. The method of claims 81-99, wherein the toxin inhibits protein synthesis.

104. The method of claims 81-103, wherein the target tissue comprises bone marrow tissue.

105. The method of claims 81-92, wherein the subject has a malignant, pre-malignant or non-malignant disorder.

106. The method of claims 81-92, wherein the subject has a disorder selected from the group consisting of glycogen storage diseases, mucopolysccharidoses, Gaucher's Disease, Hurlers Disease, sphingolipidoses, metachromatic leukodystrophy, severe combined immunodeficiency, Wiscott-Aldrich syndrome, hyper IGM syndrome, Chédiak-Higashi disease, hereditary lymphohistiocytosis, osteopetrosis, osteogenesis imperfect, a storage disease, thalassemia major, sickle cell disease, systemic sclerosis, systemic lupus erythematosus, multiple sclerosis, and juvenile rheumatoid arthritis.

107. The method of claims 81-92, wherein the subject has or is affected by a malignancy selected from the group consisting of leukemia, lymphoma, multiple myeloma, myelodysplastic syndrome and neuroblastoma.

108. The method of claims 81-107, wherein the factor is the lethal factor N-terminus (LFN) or a fragment thereof.

109. The method of claims 81-107, wherein the factor is edema factor N-terminus (EFN) or a fragment thereof.

110. The method of claims 1-23, 72-77 and 82-98, wherein the toxin comprises an RNA polymerase II and/or III inhibitor.

111. The method of claim 110, wherein the RNA polymerase II and/or III inhibitor comprises an amatoxin.

112. The method of claim 111, wherein the amatoxin is selected from the group consisting of α-amanitin, β-amanitin, γ-amanitin, £-amanitin, amanin, amaninamide, amanullin, amanullinic acid and any functional fragments, derivatives or analogs thereof.

113. The method of claims 1-23, 72-77 and 82-98, wherein the toxin comprises a DNA-damaging molecule.

114. The method of claim 113, wherein the DNA-damaging molecule is selected from the group consisting of an anti-tubulin agent, a DNA crosslinking agent, a DNA alkylating agent and a mitotic disrupting agent.

115. The method of claim 113, wherein the DNA-damaging molecule comprises maytansine or a functional fragments, derivatives or analogs thereof.

116. The method of claims 1-115, wherein the ratio of agent to toxin is about 1:1.

117. The method of claims 1-115, wherein the ratio of agent to toxin is about 4:1.

118. The method of claims 1-115, wherein the agent is bispecific.

119. The method of claim 1-115, further comprising administering to the subject one or more mobilization agents.

120. The method of claim 119, wherein the mobilizing agent is selected from the group consisting of a filgrastim, CXCR2 agonist, a CXCR4 antagonist and combinations thereof

121. The method of claim 119, wherein the mobilizing agent comprises Gro-beta.

122. The method of claim 119, wherein the mobilizing agent comprises Gro-betaΔ4.

123. The method of claims 118-122, wherein the mobilizing agent comprises plerixafor.

124. The method of claims 81-123, wherein the hematopoietic stem cells or progenitor cells express one or more markers selected from the group of markers consisting of HLA-DR, CD11a, CD18, CD34, CD41/61, CD43, CD58, CD71, CD97, CD162, CD166, CD205 and CD361

125. The method of claims 81-123, wherein the hematopoietic stem cell or progenitor cell population expresses one or more markers selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L.

126. The method of claims 81-123, wherein the markers are selected from the group consisting of CD51/61, CD72, CD45RA, CD107a, CD45RB, CD7, CD13, CD132 and CD321.

127. The method of claims 81-123, wherein the agent comprises an antibody selected from the group consisting of clone KPL-1, clone 1G10, clone M-A712, clone B6H12, clone VIM3b, clone MG38, clone G46-6 (L243), clone 581, clone 9F10, clone 12G5, clone 2G7, clone T U145, clone G43-25B and clone Dreg 56.

128. The method of claims 81-123, wherein the agent is an antibody comprising clone 1G10.

129. The method of claims 81-123, wherein the agent is an antibody comprising clone B6H12.

130. The method of claims 81-123, wherein the agent comprises an antibody selected from the group consisting of clone 23C6, clone J4-117, clone HI100, clone H4A3, clone MT4, clone M-T701, clone WM15, clone TUGh4 and clone M.AB.F11

131. The method of claims 81-123, wherein the agent comprises an antibody selected from the group consisting of clone TU39, clone TU99, clone N6B6, clone TU41, clone UM7F8, clone H5C6, clone G44-26, clone G46-2.6, clone HECA-452, clone CBR-1C2/2.1, clone 1C3, clone EBA-1, clone HIM6, clone p282 (H19), clone AK-4, clone CSLEX1, clone G28-8, clone 11G7, clone VC5, clone 28D4, clone 3A6, clone 2D7/CCR5, clone SN2, clone TU169, clone WM59, clone GHI/75, clone 9F5, clone HIP2, clone FN50, clone KPL-1, clone 1G10, clone M-A712, clone B6H12, clone VIM3b, clone MG38, clone G46-6 (L243), clone 581, clone 9F10, clone 12G5, clone 2G7, clone TU145, clone G43-25B and clone Dreg 56.

132. The method of claims 81-123, wherein the agent comprises an antibody, and wherein the antibody comprises a complementarity determining region that is the same as the complementarity determining region for one or more antibodies selected from the group consisting of clone 23C6, clone J4-117, clone HI100, clone H4A3, clone MT4, clone M-T701, clone WM15, clone TUGh4 and clone M.AB.F11

133. The method of claims 81-123, wherein the agent comprises an antibody, and wherein the antibody comprises a complementarity determining region that is the same as the complementarity determining region for one or more antibodies selected from the group consisting of clone TU39, clone TU99, clone N6B6, clone TU41, clone UM7F8, clone H5C6, clone G44-26, clone G46-2.6, clone HECA-452, clone CBR-1C2/2.1, clone 1C3, clone EBA-1, clone HIM6, clone p282 (H19), clone AK-4, clone CSLEX1, clone G28-8, clone 11G7, clone VC5, clone 28D4, clone 3A6, clone 2D7/CCR5, clone SN2, clone TU169, clone WM59, clone GHI/75, clone 9F5, clone HIP2, clone FN50, clone KPL-1, clone 1G10, clone M-A712, clone B6H12, clone VIM3b, clone MG38, clone G46-6 (L243), clone 581, clone 9F10, clone 12G5, clone 2G7, clone TU145, clone G43-25B and clone Dreg 56.

134. The method of claims 81-98, wherein the toxin is selected from the group of toxins consisting of abrin toxin, modeccin toxin, gelonin toxin, momordin toxin, trichosanthin toxin, luffin toxin, Shiga-like toxin chain A, bouganin and combinations thereof.

135. The method of claims 81-134 wherein the subject is in need of induction of solid organ transplant tolerance.

136. An immunotoxin composition comprising an agent and a toxin, wherein the agent is coupled to the toxin, wherein the agent is selected from the group consisting of an antibody and a ligand, and wherein the agent selectively binds to one or more markers expressed on human hematopoietic stem cells or progenitor cells, wherein the markers are selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a, CD62L, CD51/61, CD72, CD45RA, CD107a, CD45RB, CD7, CD13, CD132 and CD321.

137. The immunotoxin composition of claim 136, wherein the agent comprises an antibody.

138. The immunotoxin composition of claims 136 and 137, wherein the markers are selected from the group consisting of HLA-DR, HLA-DP, HLA-DQ, β2-microglobulin, CD164, CD50, CD98, CD63, CD44, HLA-A, HLA-B, HLA-C, CLA, CD102, CD58, CD326, CD147, CD59, CD62P, CD15s, CD180, CD282, CD49e, CD140b, CD166, CD195, CD165, CD31, CD85, CD123, CD41b, CD69, CD162, CD43, CD71, CD47, CD97, CD205, CD34, CD49d, CD184, CD84, CD48, CD11a and CD62L.

139. The immunotoxin composition of claims 136 and 137, wherein the markers are selected from the group consisting of CD51/61, CD72, CD45RA, CD107a, CD45RB, CD7, CD13, CD132 and CD321.

140. The immunotoxin composition of claims 136-139, wherein the agent is an antagonist of the marker.

141. The immunotoxin composition of claims 136-140, wherein the agent is not an antagonist of the marker.

142. The immunotoxin composition of claims 136-141, wherein the toxin is selected from the group of toxins consisting of saporin, diphtheria toxin, pseudomonas exotoxin A, Ricin A chain derivatives, Shiga-like toxin chain A, bouganin, a small molecule toxin and combinations thereof.

143. The immunotoxin composition of claims 136-141, wherein the toxin is selected from the group of toxins consisting of abrin toxin, modeccin toxin, gelonin toxin, momordin toxin, trichosanthin toxin, luffin toxin, Shiga-like toxin chain A, bouganin and combinations thereof.

144. The immunotoxin composition of claims 136-141, wherein the toxin comprises an RNA polymerase II and/or III inhibitor.

145. The immunotoxin composition of claim 144, wherein the RNA polymerase II and/or III inhibitor comprises an amatoxin.

146. The immunotoxin composition of claim 145, wherein the amatoxin is selected from the group consisting of α-amanitin, β-amanitin, γ-amanitin, £-amanitin, amanin, amaninamide, amanullin, amanullinic acid and any functional fragments, derivatives or analogs thereof.

147. The immunotoxin composition of claims 136-146, wherein the toxin comprises a DNA-damaging molecule.

148. The immunotoxin composition of claim 147, wherein the DNA-damaging molecule is selected from the group consisting of an anti-tubulin agent, a DNA crosslinking agent, a DNA alkylating agent and a mitotic disrupting agent.

149. The immunotoxin composition of claim 147, wherein the DNA-damaging molecule comprises maytansine or a functional fragments, derivatives or analogs thereof.

150. The immunotoxin composition of claims 136-144, wherein the toxin comprises saporin.

151. The immunotoxin composition of claims 136-144, wherein the toxin inactivates ribosomes.

152. The immunotoxin composition of claims 136-144, wherein the toxin inhibits protein synthesis.

153. The immunotoxin composition of claims 136-144, wherein the toxin is not a radioimmunotoxin.

154. The immunotoxin composition of claims 136-153, wherein the agent is directly coupled to the toxin.

155. The immunotoxin composition of claims 136-153, wherein the agent is indirectly coupled to the toxin.

156. The immunotoxin composition of claim 155, wherein the agent is biotinylated.

157. The immunotoxin composition of claim 155, wherein the agent is coupled to a streptavidin-toxin chimera.

158. The immunotoxin composition of claims 136-157, wherein the ratio of agent to toxin is about 1:1.

159. The immunotoxin composition of claims 136-157, wherein the ratio of agent to toxin is about 4:1.
